(19) United States
(12) Patent Application Publication
(10) Pub. No.: US 2024/0140920 A1
(43) Pub. Date: May 2, 2024

(54) SYNTHESIS OF URIDINE

(71) Applicant: Merck Sharp & Dohme LLC, Rahway, NJ (US)

(72) Inventors: Tamas Benkovics, East Brunswick, NJ (US); Anna Fryszkowska, New York, NY (US); Mark A. Huffman, Warren, NJ (US); Tetsuji Itoh, Somerset, NJ (US); Kevin M. Maloney, Metuchen, NJ (US); John McIntosh, Metuchen, NJ (US); Grant S. Murphy, Princeton, NJ (US)

(73) Assignee: Merck Sharp & Dohme LLC, Rahway, NJ (US)

(21) Appl. No.: 18/264,586

(22) PCT Filed: Dec. 17, 2021
(86) PCT No.: PCT/US2021/064014

§ 371 Date: Aug. 8, 2023

Related U.S. Application Data
(60) Provisional application No. 63/148,357, filed on Feb. 11, 2021.

(51) Int. Cl. C07D 273/04

(52) U.S. Cl. CPC C07D 273/04

(57) ABSTRACT

The present invention relates to efficient synthetic processes useful in the preparation of uridine, which is useful in the production of nucleosides and nucleotides that may be active as antiviral agents, as well as compositions and methods thereof.

### FIELD OF THE INVENTION

The present invention relates to efficient synthetic processes useful in the preparation of uridine, which may be useful in the preparation of other nucleosides and nucleotides, which in turn may be useful as antiviral agents.

### BACKGROUND OF THE INVENTION

Uridine, one of the nucleosides that make up ribonucleic acids, is an important biochemical compound involved in RNA synthesis, cell membrane formation, and glycosylation. See Dobolyi et al., Uridine Function in the Central Nervous System, 11(8) CURRENT TOPICS IN MEDICINAL CHEMISTRY 1058-1067 (2011). Uridine is also widely used in the pharmaceutical industry as a precursor in drug substance synthesis, such as in the synthesis of the antiviral drug molnupiravir (Iglesias et al., Biocatalytic Approaches Applied to the Synthesis of Nucleoside Prodrugs, 33(5) BIOTECHNOLOGY ADVANCES 412-434 (2015)). Although it is abundant in nature, chemical manufacturing of uridine is important for ensuring supply. Various manufacturing processes have been reported, including chemical syntheses that use multiple steps (17(1) Chinese Journal of Medicinal Chemistry 37-40 (2007); Chinese Patent Application Publication No. CN103709220A; US Patent Application Publication No. US2003/0153745A1), chemical syntheses that are accomplished in one step (Chinese Patent Application Publication No. CN101717420A), and biocatalytic syntheses (Japanese Patent Application Publication No. JP2015160832A; Ding et al., Enzymatic Synthesis of Nucleosides by Nucleoside Phosphorylase Co-Expressed in Escherichia coli, 11(11) J. Zhejiang Univ-Sci B (Biomed & Biotechnol) 880-888 (2010)).

Chemical synthesis of uridine by known routes generally requires multiple chemical steps that have harsh reaction conditions and use “non-green” chemicals. The available biocatalytic syntheses are limited to small scale preparation of uridine under diluted reaction conditions.

However, there is a need for synthetic routes to prepare uridine that are efficient, sustainable, and employ green chemistry.

### SUMMARY OF THE INVENTION

The present invention relates to processes useful in the synthesis of uridine. The processes of the present invention afford advantages over previously known procedures and include a more efficient route to uridine.

Other embodiments, aspects and features of the present invention are either further described in or will be apparent from the ensuing description, examples and appended claims.

### DETAILED DESCRIPTION OF THE INVENTION

### Abbreviations

g/L grams per liter
h hour
mg milligram
min minutes
ml, mL milliliter
mM millimolar, 1 mM is a concentration of one thousandth of a mole per liter
mmol millimole, a thousandth of a mole (the amount of any chemical substance that equals the number of atoms in 12 grams of carbon-12, approximately 6.022140857×10²³).
N Normality, the gram equivalent weight of a solution in a solution, which is its molar concentration divided by an equivalence factor.
rpm Revolutions per minute
ul, uL, μl, μL Microliter
μmol micromole, a 10⁻⁶ (one-millionth) of a mole

Additional abbreviations may be defined throughout this disclosure.

### Definitions

Certain technical and scientific terms are specifically defined below. Unless specifically defined elsewhere in this document, all other technical and scientific terms used herein have the meaning commonly understood by one of ordinary skill in the art to which this disclosure relates. That is, terms used herein have their ordinary meaning, which is independent at each occurrence thereof. That notwithstanding and except where stated otherwise, the following definitions apply throughout the specification and claims. In case of conflict, the present specification, including definitions, will control. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

As used herein, including the appended claims, the singular forms of words, such as “a,” “an,” and “the,” include their corresponding plural references unless the context clearly dictates otherwise. In particular, “a,” “an,” and “the” item each include a single item selected from a list as well as mixtures of two or more items selected from the list.

As used herein, the terms “at least one” item or “one or more” item each include a single item selected from the list as well as mixtures of two or more items selected from the list. For example, “at least one S-methyl-5-thioribose kinase enzyme” (alternatively referred to as “S-methyl-5-thioribose kinase enzymes,” “at least one MTR kinase,” “MTR kinases,” “at least one MTR kinase enzyme,” or “MTR kinase enzymes”) refers to a single MTR kinase as well as to mixtures of two or more different MTR kinases. Similarly, the terms “at least two” items and “two or more” items each include mixtures of two items selected from the list as well as mixtures of three or more items selected from the list.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, and ±10%, and their numerical equivalents. “About” when used to modify a numerically defined parameter (e.g., the dose of an antiviral nucleoside, or the length of treatment time with a combination therapy described herein) means that the parameter may vary by as much as 10% below or above the stated numerical value for that parameter; where appropriate, the stated parameter may be rounded to the nearest whole number. For example, a dose of about 5 mg/kg may vary between 4.5 mg/kg and 5.5 mg/kg. In addition, the term “or,” as used herein, denotes alternatives that may, where appropriate, be combined; that is, the term “or” includes each listed alternative separately as well as their combination.

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl, and neohexyl. In one embodiment, an alkyl group is linear. In another embodiment, an alkyl group is branched.

The terms “halogen” and “halo,” as used herein, means —F (fluorine), —Cl (chlorine), —Br (bromine), or —I (iodine).

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group has from 1 to 3 carbon atoms. In another embodiment, a haloalkyl group is substituted with from 1 to 3 halogen atoms. Non-limiting examples of haloalkyl groups include —CH₂F, —CHF₂, and —CF₃. The term “C₁-C₄ haloalkyl” refers to a haloalkyl group having from 1 to 4 carbon atoms.

The term “alkoxy” as used herein, refers to an —O -alkyl group, wherein an alkyl group is as defined above. Non-limiting examples of alkoxy groups include methoxy, ethoxy, n-propoxy, isopropoxy, n-butoxy, and tert-butoxy. An alkoxy group is bonded via its oxygen atom to the rest of the molecule.

The term “aryl,” as used herein, refers to an aromatic monocyclic or multicyclic ring system comprising from about 6 to about 14 carbon atoms. In one embodiment, an aryl group contains from about 6 to about 10 carbon atoms (C₆-C₁₀ aryl). In another embodiment an aryl group is phenyl. Non-limiting examples of aryl groups include phenyl and naphthyl.

When a functional group in a compound is termed “protected,” the group is in modified form to preclude undesired side reactions at the protected site when the compound is subjected to a reaction. The term “PG”, as used herein, refers to a protecting group. Those skilled in the art will readily envisage protecting groups (PG) suitable for use in compounds and processes according to the disclosure. Suitable protecting groups will be recognized by those of ordinary skill in the art as well as by reference to standard textbooks such as, for example, T. W. Greene et al., Protective Groups in Organic Synthesis (1991), Wiley, New York. Protecting groups suitable for use herein include acid-labile protecting groups. Non-limiting examples of PG suitable for use herein include —S(O)₂R⁸, —C(O)OR⁸, —C(O)R⁸, —CH₂OCH₂CH₂SiR⁸, and —CH₂R₈, wherein R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently is selected from the group consisting of —OCH₃, —Cl, —Br, and —I.

The term “substituted” means that one or more hydrogens on the atoms of the designated moiety are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

When any substituent or variable occurs more than one time in any compound, its definition on each occurrence is independent of its definition at every other occurrence, unless otherwise indicated. For example, description of radicals that include the expression “—N(C₁-C₃ alkyl)₂” means —N(CH₃)(CH₂CH₃), —N(CH₃)(CH₂CH₂CH₃), and —N(CH₂CH₃)(CH₂CH₂CH₃), as well as —N(CH₃)₂, —N(CH₂CH₃)₂, and —N(CH₂CH₂CH₃)₂.

It should also be noted that any carbon or heteroatom with unsatisfied valences in the text, schemes, examples and Tables herein is assumed to have sufficient hydrogen atom(s) to satisfy the valences. Any one or more of these hydrogen atoms can be deuterium.

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²³I, respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half-lives (T_{1/2}>1 day), can generally be prepared by following procedures analogous to those disclosed in the Schemes and/or in the Examples herein below, by substituting an appropriate isotopically labeled reagent for a non-isotopically labeled reagent.

One or more compounds herein may exist in unsolvated as well as solvated forms with pharmaceutically acceptable solvents, such as water, ethanol, and the like, and this disclosure is intended to embrace both solvated and unsolvated forms. “Solvate” means a physical association of a compound with one or more solvent molecules. This physical association involves varying degrees of ionic and covalent bonding, including hydrogen bonding. In certain instances of this aspect, the solvate will be capable of isolation, for example when one or more solvent molecules are incorporated in the crystal lattice of a crystalline solid. “Solvate” encompasses both solution-phase and isolatable solvates. Non-limiting examples of suitable solvates include ethanolates, methanolates, and the like. “Hydrate” is a solvate in which the solvent molecule is H₂O.

Compounds herein may contain one or more stereogenic centers and can thus occur as racemates, racemic mixtures, single enantiomers, diastereomeric mixtures, and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates, and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the S or R configuration as defined by the IUPAC 1974 Recommendations.

The present disclosure further includes compounds and synthetic intermediates in all their isolated forms. For example, the identified compounds are intended to encompass all forms of the compounds such as, any solvates, hydrates, stereoisomers, and tautomers thereof.

Those skilled in the art will recognize that certain compounds, and in particular compounds containing certain heteroatoms and double or triple bonds, can be tautomers, structural isomers that readily interconvert. Thus, tautomeric compounds can be drawn in a number of different ways that are equivalent. Non-limiting examples of such tautomers include those exemplified below.

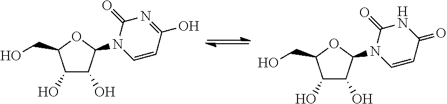 [SMILES: O=c1nc(O)ccn1[C@@H]1O[C@H](CO)[C@@H](O)[C@H]1O ; O=c1ccn([C@@H]2O[C@H](CO)[C@@H](O)[C@H]2O)c(=O)[nH]1]

Those skilled in the art will recognize that chiral compounds, and in particular sugars, can be drawn in a number of different ways that are equivalent. Those skilled in the art will further recognize that the identity and regiochemical position of the substituents on ribose can vary widely and that the same principles of stereochemical equivalence apply regardless of substituent. Non-limiting examples of such equivalence include those exemplified below.

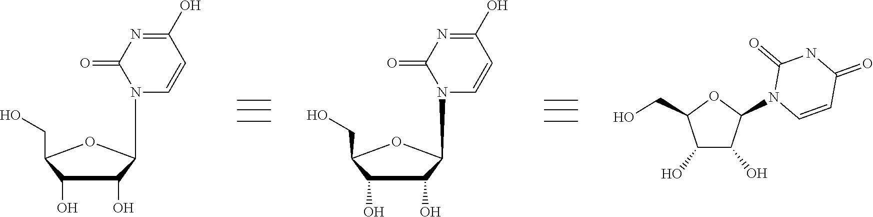

Compounds can form salts that are also within the scope of this disclosure. Reference to a compound herein is understood to include reference to salts thereof, unless otherwise indicated. The term “salt(s),” as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,), and the like. Additionally, acids that are generally considered suitable for the formation of pharmaceutically useful salts from basic pharmaceutical compounds are discussed, for example, by P. Stahl et al., Camille G. (eds.) Handbook of Pharmaceutical Salts: Properties, Selection and Use (2002) Zurich: Wiley-VCH; S. Berge et al., J. Pharm. Sci. (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C.). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, tert-butyl amines, and salts with amino acids such as arginine, lysine, and the like. Basic nitrogen-containing groups may be quarternized with agents such as lower alkyl halides (e.g. methyl, ethyl, and butyl chlorides, bromides, and iodides), dialkyl sulfates (e.g. dimethyl, diethyl, and dibutyl sulfates), long chain halides (e.g. decyl, lauryl, and stearyl chlorides, bromides, and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristoylation, ubiquitination, etc.). Included within this definition are D- and L-amino acids, and mixtures of D- and L-amino acids, as well as polymers comprising D- and L-amino acids, and mixtures of D- and L-amino acids. Proteins, polypeptides, and peptides may include a tag, such as a histidine tag, which should not be included when determining percentage of sequence identity.

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of enzymes, identifies the originating enzyme, and/or the gene encoding such enzyme, upon which the enzyme was based. For example, the MTR kinase of SEQ ID NO: 4 was obtained by artificially evolving, over multiple generations the gene encoding the MTR kinase enzyme of SEQ ID NO: 1. Thus, this evolved MTR kinase enzyme is “derived from” the MTR kinase of SEQ ID NO: 1.

“Hydrophilic amino acid or residue” refers to an amino acid or residue having a side chain exhibiting a hydrophobicity of less than zero according to the normalized consensus hydrophobicity scale of Eisenberg et al., 1984, J. MOL. BIOL. 179:125-142. Genetically encoded hydrophilic amino acids include L-Thr (T), L-Ser (S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S), and L-Thr (T).

“Hydrophobic amino acid or residue” refers to an amino acid or residue having a side chain exhibiting a hydrophobicity of greater than zero according to the normalized consensus hydrophobicity scale of Eisenberg et al., 1984, J. MOL. BIOL. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five-membered ring.

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with -SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T), and L-Asp (D).

“Hydroxyl-containing amino acid or residue” refers to an amino acid containing a hydroxyl (—OH) moiety. Genetically-encoded hydroxyl-containing amino acids include L-Ser (S) L-Thr (T), and L-Tyr (Y).

As used herein, “polynucleotide” and “nucleic acid” refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

As used herein, “nucleoside” refers to glycosylamines comprising a nucleobase (i.e., a nitrogenous base), and a 5-carbon sugar (e.g., ribose or deoxyribose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups. In some embodiments, nucleosides can be phosphorylated by kinases to produce nucleotides.

As used herein, “nucleoside diphosphate” refers to glycosylamines comprising a nucleobase (i.e., a nitrogenous base), a 5-carbon sugar (e.g., ribose or deoxyribose), and a diphosphate (i.e., pyrophosphate) moiety. In some embodiments herein, “nucleoside diphosphate” is abbreviated as “NDP.” Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). The terms “nucleoside” and “nucleotide” may be used interchangeably in some contexts.

As used herein, “nucleoside triphosphate” refers to glycosylamines comprising a nucleobase (i.e., a nitrogenous base), a 5-carbon sugar (e.g., ribose or deoxyribose), and a triphosphate moiety. In some embodiments herein, “nucleoside triphosphate” is abbreviated as “NTP.” Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). The terms “nucleoside” and “nucleotide” may be used interchangeably in some contexts.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition) and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the wild-type enzyme, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.5 times the enzymatic activity of the corresponding wild-type enzyme, to as much as 2 times. 5 times, 10 times, 20 times, 25 times, 50 times, 75 times, 100 times, 150 times, 200 times, 500 times, 1000 times, 3000 times, 5000 times, 7000 times, or more enzymatic activity than the naturally occurring enzyme or another enzyme from which the polypeptides were derived. In specific embodiments, the enzyme exhibits improved enzymatic activity in the range of 150 to 3000 times, 3000 to 7000 times, or more than 7000 times greater than that of the parent enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹s⁻¹). Hence, any improvements in the enzyme activity will have an upper limit related to the diffusion rate of the substrates acted on by the enzyme. Enzyme activity can be measured by any one of standard assays used for measuring kinase activity, or via a coupled assay with a nucleoside phosphorylase enzyme which is capable of catalyzing reaction between the polypeptide product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, or 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation, with the sole exception that wild-type polypeptide or polynucleotide sequences as identified herein may include a tag, such as a histidine tag, which should not be included when determining percentage of sequence identity. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. MOL. BIOL. 215: 403-410; and Altschul et al., 1977, NUCLEIC ACIDS RES. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. SCI. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, ADV. APPL. MATH. 2:482, by the homology alignment algorithm of Needleman and Wunsch, 1970, J. MOL. BIOL. 48:443, by the search for similarity method of Pearson and Lipman, 1988, N USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30 to 50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to”, or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (EE) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereomeric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to a polypeptide that maintains similar activity (more than 60% to 80% for example) after exposure to elevated temperatures (e.g., 40° C. to 80° C.) for a period of time (e.g., 0.5 h to 24 h) compared to the untreated enzyme.

“Solvent stable” refers to a polypeptide that maintains similar activity (more than e.g., 60% to 80%) after exposure to varying concentrations (e.g., 5 h to 99%) of solvent (isopropyl alcohol, tetrahydrofuran, 2-methyltetrahydrofuran, acetone, toluene, butylacetate, methyl tert-butylether, etc.) for a period of time (e.g., 0.5 h to 24 h) compared to the untreated enzyme.

“pH stable” refers to a polypeptide that maintains similar activity (more than e.g., 60% to 80%) after exposure to high or low pH (e.g., 4.5 to 6 or 8 to 12) for a period of time (e.g., 0.5 h to 24 h) compared to the untreated enzyme.

“Thermo- and solvent stable” refers to a polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

Exemplary methods and materials are described herein, although methods and materials similar or equivalent to those described herein can also be used in the practice or testing of the present disclosure. The materials, methods, and examples are illustrative only and not intended to be limiting.

### Processes for Preparing Uridine

The present disclosure provides enzymatic processes for preparing uridine, and pharmaceutically acceptable salts, hydrates, and solvates thereof:

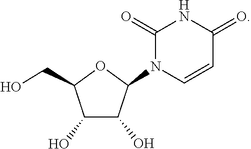 [SMILES: O=c1ccn([C@@H]2O[C@H](CO)[C@@H](O)[C@H]2O)c(=O)[nH]1]

In embodiments, the disclosure provides enzymatic processes for preparing uridine, and hydrates, and solvates thereof.

In embodiments, the processes of the disclosure may be conducted in a single vessel, as a “one-pot” process, or the steps may be conducted sequentially. In embodiments, the intermediate products may optionally be isolated.

In a first embodiment of the processes of the disclosure, the process comprises reacting ribose with uracil in the presence of at least one enzyme to form uridine:

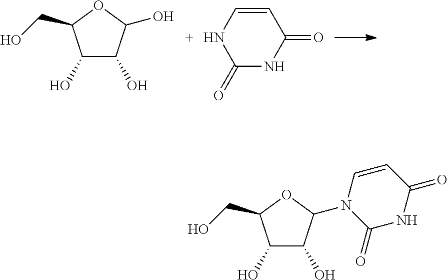 [SMILES: OC[C@H]1OC(O)[C@H](O)[C@@H]1O ; O=c1cc[nH]c(=O)[nH]1 ; O=c1ccn(C2O[C@H](CO)[C@@H](O)[C@H]2O)c(=O)[nH]1]

In a first aspect of this first embodiment, uracil is provided in an amount in a range of from about 0.4 to about 1.2 equivalents with respect to the amount of ribose, such as an amount of about 0.8 equivalents.

In a second aspect of this first embodiment, the at least one enzyme (alternatively “an enzyme”) is selected from the group consisting of at least one S-methyl-5-thioribose kinase enzyme (alternatively referred to as “S-methyl-5-thioribose kinase enzymes”), at least one acetate kinase enzyme (alternatively referred to as “acetate kinase enzymes”), at least one pyruvate oxidase enzyme (alternatively referred to as “pyruvate oxidase enzymes”), at least one catalase enzyme (alternatively referred to as “catalase enzymes”), at least one uridine phosphorylase enzyme (alternatively referred to as “uridine phosphorylase enzymes”), at least one ribokinase enzyme (alternatively referred to as “ribokinase enzymes”), at least one phosphopentomutase enzyme (alternatively referred to as “phosphopentomutase enzymes”), and at least one sucrose phosphorylase enzyme (alternatively referred to as “sucrose phosphorylase enzymes”), and mixtures thereof.

In instances of this second aspect, the at least one enzyme is a mixture of at least two enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In some instances, the at least one enzyme is a mixture of at least three enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In further instances, the at least one enzyme is a mixture of at least four enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In still further instances, the at least one enzyme is a mixture of at least five enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme. In particular instances, the at least one enzyme is a mixture of at least one S-methyl-5-thioribose kinase enzyme, at least one acetate kinase enzyme, at least one pyruvate oxidase enzyme, at least one catalase enzyme, and at least one uridine phosphorylase enzyme. In other particular instances, the at least one enzyme is a mixture of at least one S-methyl-5-thioribose kinase enzyme, at least one acetate kinase enzyme, at least one uridine phosphorylase enzyme, and at least one sucrose phosphorylase enzyme. In additional particular instances, the at least one enzyme is a mixture of at least one acetate kinase enzyme, at least one pyruvate oxidase enzyme, at least one catalase enzyme, at least one uridine phosphorylase enzyme, at least one ribokinase enzyme, and at least one phosphopentomutase enzyme. In further particular instances, the at least one enzyme is a mixture of at least one S-methyl-5-thioribose kinase enzyme, at least one acetate kinase enzyme, at least one uridine phosphorylase enzyme, at least one ribokinase enzyme, at least one phosphopentomutase enzyme, and at least one sucrose phosphorylase enzyme. In still further particular instances, the at least one enzyme is a mixture of at least one acetate kinase enzyme, at least one uridine phosphorylase enzyme, and at least two enzymes selected from the group consisting of at least one S-methyl-5-thioribose kinase enzyme, at least one pyruvate oxidase enzyme, at least one catalase enzyme, at least one ribokinase enzyme, at least one phosphopentomutase enzyme, and at least one sucrose phosphorylase enzyme.

In a first instance of the second aspect, the at least one S-methyl-5-thioribose kinase enzyme is one or more S-methyl-5-thioribose kinase selected from the group consisting of wild-type S-methyl-5-thioribose kinase enzymes and S-methyl-5-thioribose kinase enzymes that are produced from the directed evolution from a commercially available, wild-type S-methyl-5-thioribose kinase enzymes. In specific instances, the at least one S-methyl-5-thioribose kinase enzyme is selected from the group consisting of S-methyl-5-thioribose kinase enzymes that are produced from the directed evolution from a commercially available, wild-type S-methyl-5-thioribose kinase enzyme, which has the amino acid sequence as set forth below in SEQ ID NO: 1.

(SEQ ID NO: 1)
MSQYHTFTAHDAVAYAQQFAGIDNPSELVSAQEVGDGNLNLVFKVFDRQG
VSRAIVKQALPYVRCVGESWPLTLDRARLEAQTLVAHYQHSPQHTVKIHH
FDPELAVMVMEDLSDHRIWRGELIANVYYPQAARQLGDYLAQVLFHTSDF
YLHPHEKKAQVAQFINPAMCEITEDLFFNDPYQIHERNNYPAELEADVAA
LRDDAQLKLAVAALKHRFFAHAEALLHGDIHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYS

In occurrences, the at least one S-methyl-5-thioribose kinase enzyme is the wild-type S-methyl-5-thioribose kinase having the amino acid sequence as set forth above in SEQ ID NO: 1. In specific occurrences, the wild-type S-methyl-5-thioribose kinase may be encoded by the DNA sequence as set forth below in SEQ ID NO: 2.

(SEQ ID NO: 2)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACGTGCGTTGCGTTGG
TGAAAGCTGGCCGCTGACCCTGGACCGTGCGCGTCTGGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCACCGAAGACCTGTTCTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACATTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCTAA

In specific occurrences of this first instance, the at least one S-methyl-5-thioribose kinase enzyme comprises the amino acid sequence as set forth below in SEQ ID NO: 3.

(SEQ ID NO: 3)
MSQYHTFTAHDAVAYAQQFAGIDNPSELVSAQEVGDGNLNLVFKVFDRQG
VSRAIVKQALPYVRCVGESWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEITEDLFFNDPYQIHERNNYPAELEADVAA
LRDDAQLKLAVAALKHRFFAHAEALLHGDIHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 3 may be encoded by the DNA sequence as set forth below in SEQ ID NO: 4.

(SEQ ID NO: 4)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACGTGCGTTGCGTTGG
TGAAAGCTGGCCGCTGACCCTGGACCGTGCGCGTCATGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCACCGAAGACCTGTTCTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACATTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCCTC
GAGCACCACCACCACCACCACTGA

In specific occurrences of this first instance, the at least one S-methyl-5-thioribose kinase enzyme comprises the amino acid sequence as set forth below in SEQ ID NO: 5.

(SEQ ID NO: 5)
MSQYHTFTAHDAVAYAQQFAGIDNPSELVSAQEVGDGNLNLVFKVFDRQG
VSRAIVKQALPYPRCVGESWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEISEDLIFNDPYQIHERNNYPAELEADVAA
LRDDAQLKLAVAALKHRFFAHAEALLHGDLHSGSIFVAEGSLKAIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 5 may be encoded by the DNA sequence as set forth below in SEQ ID NO: 6.

(SEQ ID NO: 6)
ATGAGCCAGTATCATACCTTCACCGCGCATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACCCCCGTTGCGTTGG
TGAAAGCTGGCCGCTGACCCTGGACCGTGCGCGTCATGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCGATGTGCGAGATCAGCGAAGACCTGATTTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACCTTCACAGCGGCA
GCATCTTCGTTGCGGAGGGTAGCCTGAAGGCGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGAGC
CACGTTGCGGACATCGATACCATTCAGGACGATGCGATGCGTCACGAATG
CCTGCGTCACGCGATCACCCTGGGTCGTGCGCTGATTGTTCTGGCGGAGC
GTATCGACAGCGTGGATGAACTGCTGGCGCGTGTTCGTCAATACAGCCTC
GAGCACCACCACCACCACCACTGA

In specific occurrences of this instance, the at least one S-methyl-5-thioribose kinase enzyme comprises the amino acid sequence as set forth below in SEQ ID NO: 7.

(SEQ ID NO: 7)
MSQYHTFTADDAVAYAQQFAGIDNPSELVSAQEVGDGNLNLVFKVFDRQG
VSRAIVKQALPYPRRVGPSWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPAMCEISEDLVFNDPYQIHERNNYPAELEADVAA
LRDDAQLKLAVAALKHRFFAHAEALLHGDLHTGSIFVAEGSLKVIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLA
HVADIDTIQDDAMRHECLREAITLGRALIVLAERIDSVDELLARVRQYSL
EHHHHHH

In specific examples of such occurrences, the S-methyl-5-thioribose kinase enzyme comprising the amino acid sequence as set forth above in SEQ ID NO: 7 may be encoded by the DNA sequence as set forth below in SEQ ID NO: 8.

(SEQ ID NO: 8)
ATGAGCCAGTATCATACCTTCACCGCGGATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACCCCCGTCGCGTTGG
TCCGAGCTGGCCGCTGACCCTGGACCGTGCCCGTCATGAAGCGCAGACCC
TGGTTGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGCCATGTGCGAAATCAGCGAAGACCTGGTGTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACCTTCACACCGGCA
GCATCTTCGTTGCGGAGGGTAGCCTGAAGGTGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGGCG
CACGTTGCGGACATCGATACCATTCAGGACGATGCGATGCGTCACGAATG
CCTGCGTGAAGCGATCACCCTGGGTCGTGCGCTGATTGTTCTGGCGGAGC
GCATCGACAGCGTGGATGAACTGCTGGCGCGTGTTCGTCAATACAGCCTC
GAGCACCACCACCACCACCACTGA

In specific occurrences of this first instance, the at least one S-methyl-5-thioribose kinase enzyme comprises the amino acid sequence as set forth below in SEQ ID NO: 9.

(SEQ ID NO: 9)
MSQYHTFTADDAVAYAQQFAGIDNPSELVSAQEVGDGNLNLVFKVFDRQG
VSRAIVKQALPYPRAVGPSWPLTLDRARHEAQTLVAHYQHSPQHTVKIHH
FDPELAVMVMEDLSDHRIWRGELIANVYYPQAARQLGDYLAQVLFHTSDF
YLHPHEKKAQVAQFINPGMCEISEDLSFNDPYQIHERNNYPAELEADVAA
LRDDAQLKLAVAALKHRFFAHAEALLHGDLHSGSIFVAEGSLKVIDAEFG
YFGPIGFDIGTAIGNLLLNYCGLPGQLGIRDAAAAREQRLNDIHQLWTTF
AERFQALAAEKTRDAALAYPGYASAFLKKVWADAVGFCGSELIRRSVGLS
HVADIDTIQDDAMRHECLRHAITLGRALIVLAETIDSVDELLARVRQYSL
EHHHHHH

In specific examples of such occurrences, the S-methyl-5-thioribose kinase enzyme comprising the amino acid sequence as set forth above in SEQ ID NO: 9 may be encoded by the DNA sequence as set forth below in SEQ ID NO: 10.

(SEQ ID NO: 10)
ATGAGCCAGTATCATACCTTCACCGCGGATGATGCGGTGGCGTATGCGCA
GCAATTTGCGGGCATTGATAACCCGAGCGAGCTGGTTAGCGCGCAAGAAG
TTGGTGACGGCAACCTGAACCTGGTGTTCAAGGTTTTTGATCGTCAGGGT
GTGAGCCGTGCGATCGTTAAACAAGCGCTGCCGTACCCCCGTGCGGTTGG
TCCGAGCTGGCCGCTGACCCTGGACCGTGCGCGTCATGAAGCGCAGACCC
TGGTGGCGCACTATCAGCACAGCCCGCAACACACCGTTAAGATCCACCAC
TTCGATCCGGAGCTGGCGGTGATGGTTATGGAAGACCTGAGCGATCACCG
TATTTGGCGTGGTGAGCTGATCGCGAACGTGTACTATCCGCAGGCGGCGC
GTCAACTGGGTGACTACCTGGCGCAGGTTCTGTTCCACACCAGCGATTTT
TATCTGCACCCGCACGAGAAGAAAGCGCAGGTGGCGCAATTCATTAACCC
GGGCATGTGCGAAATCAGCGAAGACCTGAGCTTTAACGATCCGTACCAGA
TTCACGAACGTAACAACTATCCGGCGGAGCTGGAAGCGGATGTGGCGGCG
CTGCGTGATGATGCGCAACTGAAGCTGGCGGTTGCGGCGCTGAAACACCG
TTTCTTTGCGCATGCGGAGGCGCTGCTGCATGGTGACCTTCACAGCGGCA
GCATCTTCGTTGCGGAGGGTAGCCTGAAGGTGATCGACGCGGAATTCGGT
TACTTTGGCCCGATCGGTTTTGATATTGGTACCGCGATCGGCAACCTGCT
GCTGAACTATTGCGGTCTGCCGGGTCAACTGGGTATTCGTGATGCGGCGG
CGGCGCGTGAACAGCGTCTGAACGATATCCACCAACTGTGGACCACCTTC
GCGGAGCGTTTTCAAGCGCTGGCGGCGGAAAAGACCCGTGACGCGGCGCT
GGCGTACCCGGGTTATGCGAGCGCGTTCCTGAAGAAAGTGTGGGCGGATG
CGGTTGGTTTTTGCGGCAGCGAGCTGATTCGTCGTAGCGTGGGCCTGTCG
CACGTTGCGGACATCGATACCATTCAGGACGATGCGATGCGTCACGAATG
CCTGCGTCACGCGATCACCCTGGGTCGTGCGCTGATTGTTCTGGCGGAGA
CCATCGACAGCGTGGATGAACTGCTGGCGCGTGTTCGTCAATACAGCCTC
GAGCACCACCACCACCACCACTGA

In specific occurrences of this first instance, the at least one S-methyl-5-thioribose kinase enzyme comprises the amino acid sequence as set forth below in SEQ ID NO: 11.

(SEQ ID NO: 11)
MSQYHTFTADDAVAYAQQFAGIDNPSELVSAQEVGDGNLNLVFKVFDRQ
GVSRAIVKQALPYPRRVGPSWPLTLDRARHEAQTLVAHYQHSPQHTVKI
FHFDPELAVMVMEDLSDHRIWRGELIANVYYPQAARQLGDYLAQVLFHT
SDFYLHPHEKKRQVAQFINPAMCGISEDLVENDPYQIHERNNYPAELEA
QVAALRDDAQLKLAVAALKHRFFAHAEALLHGDLHTGSIFVKEGSLKVI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLAHVADIDTIQDDAMRHECLREAITLGRALIVLAERIDSVDELL
ARVRQYSLEHHHHHH

In specific occurrences of this first instance, the at least one S-methyl-5-thioribose kinase enzyme comprises the amino acid sequence as set forth below in SEQ ID NO: 12.

(SEQ ID NO: 12)
MSQYHTFTADDAVAYAQQFAGIDNPSELVSAQEVGDGNLNLVFKVFDRQ
GVSRSIVKQALPYPRRVGPSWPLTLDRARHEAQTLVAHYQHSPQHTVKI
FHFDPELAVMVMEDLSDHRIWRGELIANVYYPQAARQLGDYLAQVLFHT
SDFYLHPHEKKRQVAQFINPAMCGISEDLVENDPYQIHERNNYPAELEA
QVAALRDDAQLKLAVAALKHRFFAHAEALLHGDLHTGSIFVKEGSLKVI
DAEFGYFGPIGFDIGTAIGNLLLNYCGLPGQLGIRDAAAAREQRLNDIH
QLWTTFAERFQALAAEKTRDAALRYPGYASAFLKKVWADAVGFCGSELI
RRSVGLAHVADIDTIQDDAMRHECLREAITLGRALIVLAERIDSVDELL
ARVRQYSLEHHHHHH

In specific occurrences of this first instance, the at least one S-methyl-5-thioribose kinase enzyme comprises the amino acid sequence as set forth below in SEQ ID NO: 13.

(SEQ ID NO: 13)
MGVTKTPLYETLNESSAVALAVKLGLFPSKSTLTCQEIGDGNLNYVFHI
YDQEHDRALIIKQAVPYAKVVGESWPLTIDRARIESSALIRQGEHVPHL
VPRVFYSDTEMAVTVMEDLSHLKIARKGLIEGENYPHLSQHIGEFLGKT
LFYSSDYALEPKVKKQLVKQFTNPELCDITERLVFTDPFFDHDTNDFEE
ELRPFVEKLWNNDSVKIEAAKLKKSFLTSAETLIHGDLHTGSIFASEHE
TKVIDPEFAFYGPIGFDVGQFIANLFLNALSRDGADREPLYEHVNQVWE
TFEETFSEAWQKDSLDVYANIDGYLTDTLSHIFEEAIGFAGCELIRRTI
GLAHVADLDTIVPFDKRIGRKRLALETGTAFIEKRSEFKTITDVIELFK
LLVKE

In specific occurrences of this first instance, the at least one S-methyl-5-thioribose kinase enzyme comprises the amino acid sequence as set forth below in SEQ ID NO: 55.

(SEQ ID NO: 55)
MSQYHTFTAHDAVAYAQQFAGIDNPSELVSAQEVGDGNLNLVFKVFDRQ
GVSRAIVKQALPYVRAVGESWPLTLDRARHEAQTLVAHYQHSPQHTVKI
HHFDPELAVMVMEDLSDHRIWRGELIANVYYPQAARQLGDYLAQVLFHT
SDFYLHPHEKKAQVAQFINPAMCEITEDLVENDPYQIHERNNYPAELEA
DVAALRDDAQLKLAVAALKHRFFAHAEALLHGDIHSGSIFVAEGSLKVI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGMSHVADIDTIQDDAMRHECLRHAITLGRALIVLAERIDSVDELL
ARVRQYSLEHHHHHH

In some occurrences, the at least one S-methyl-5-thioribose kinase enzyme is a S-methyl-5-thioribose kinase enzyme based on the amino acid sequences of SEQ ID NO: 1, 3, 5, 7, 9, 11, 12, 13, or 55 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, 12, 13, or 55. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one S-methyl-5-thioribose kinase enzyme is a S-methyl-5-thioribose kinase enzyme encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

In a second instance of the second aspect, the at least one acetate kinase enzyme is one or more acetate kinase enzymes selected from the group consisting of wild-type acetate kinase enzymes and acetate kinase enzymes that are produced from the directed evolution from a commercially available, wild-type acetate kinase enzyme. In specific instances, the at least one acetate kinase enzyme is selected from the group consisting of acetate kinase enzymes that are produced from the directed evolution from a commercially available, wild-type acetate kinase enzyme, which has the amino acid sequence as set forth below in SEQ ID NO:14.

(SEQ ID NO: 14)
MRVLVINSGSSSIKYQLIEMEGEKVLCKGIAERIGIEGSRLVHRVGDEK
HVIERELPDHEEALKLILNTLVDEKLGVIKDLKEIDAVGHRVVHGGERF
KESVLVDEEVLKAIEEVSPLAPLHNPANLMGIKAAMKLLPGVPNVAVFD
TAFHQTIPQKAYLYAIPYEYYEKYKIRRYGFHGTSHRYVSKRAAEILGK
KLEELKIITCHIGNGASVAAVKYGKCVDTSMGFTPLEGLVMGTRSGDLD
PAIPFFIMEKEGISPQEMYDILNKKSGVYGLSKGFSSDMRDIEEAALKG
DEWCKLVLEIYDYRIAKYIGAYAAAMNGVDAIVFTAGVGENSPITREDV
CSYLEFLGVKLDKQKNEETIRGKEGIISTPDSRVKVLVVPTNEELMIAR
DTKEIVEKIGR

In occurrences, the at least one acetate kinase enzyme is a wild-type acetate kinase enzyme having the amino acid sequence as set forth above in SEQ ID NO: 14. In specific examples of such occurrences, the wild-type acetate kinase enzyme comprising the amino acid sequence as set forth above in SEQ ID NO: 14 may be encoded by the DNA sequence as set forth below in SEQ ID NO: 15.

(SEQ ID NO: 15)
ATGCGTGTTCTGGTTATTAATAGCGGTAGCAGCAGCATTAAATATCAGC
TGATTGAAATGGAAGGTGAAAAAGTTCTGTGTAAAGGTATTGCAGAACG
TATTGGTATTGAAGGTAGCCGTCTGGTTCATCGTGTTGGTGATGAAAAA
CATGTTATTGAACGTGAACTGCCGGATCATGAAGAAGCACTGAAACTGA
TTCTGAATACCCTGGTTGATGAAAAACTGGGTGTTATTAAAGATCTGAA
AGAAATTGACGCAGTTGGTCATCGTGTTGTTCATGGTGGTGAACGTTTT
AAAGAAAGCGTTCTGGTTGATGAAGAAGTTCTGAAAGCAATTGAAGAAG
TTAGCCCGCTGGCACCGCTGCATAATCCGGCAAATCTGATGGGTATTAA
AGCAGCAATGAAACTGCTGCCGGGTGTTCCGAATGTTGCAGTTTTTGAT
ACCGCATTTCATCAGACCATTCCGCAGAAAGCATATCTGTATGCAATTC
CGTATGAATATTACGAAAAATACAAAATTCGTCGCTACGGTTTTCATGG
TACCAGCCATCGTTATGTTAGCAAACGTGCAGCAGAAATTCTGGGTAAA
AAACTGGAAGAACTGAAAATTATCACCTGTCATATTGGTAATGGTGCAA
GCGTTGCAGCAGTTAAATATGGTAAATGTGTTGATACCAGCATGGGTTT
TACCCCGCTGGAAGGTCTGGTTATGGGTACCCGTAGCGGTGATCTGGAT
CCGGCAATTCCGTTTTTTATTATGGAAAAAGAGGGTATTAGCCCGCAGG
AAATGTATGATATTCTGAATAAAAAAAGCGGCGTTTATGGTCTGAGCAA
AGGTTTTAGCAGCGATATGCGTGATATTGAAGAAGCAGCACTGAAAGGT
GATGAATGGTGTAAACTGGTTCTGGAAATTTATGATTACCGTATTGCAA
AATACATCGGTGCATACGCTGCAGCAATGAATGGTGTTGATGCAATTGT
TTTTACCGCAGGTGTTGGTGAAAATAGCCCGATTACCCGTGAAGATGTT
TGTAGCTATCTGGAATTTCTGGGTGTTAAACTGGATAAACAGAAAAATG
AAGAGACCATTCGTGGTAAAGAAGGTATTATTAGCACCCCGGATAGCCG
TGTTAAAGTTCTGGTTGTTCCGACCAATGAAGAACTGATGATTGCACGT
GATACCAAAGAAATTGTTGAAAAAATCGGTCGTTAA

In specific occurrences of this second instance, the at least one acetate kinase enzyme comprises the amino acid sequence as set forth below in SEQ ID NO: 16.

(SEQ ID NO: 16)
MGSHHHHHHGSRVLNINSGSSSIKYQLIEMEGEKVLCKGIAERIGIEGS
RLVHRVGDEKHVIERELPDHEEALKLILNTLVDEKLGVIKDLKEIDAVG
HRVVHGGERFKESVLVDEEVLKAIEEVSPLAPLHNPANLMGIKAAMKLL
PGVPNVQVFDTAFHQTIPQKAYLYAIPYEYYEKYKIRRYGFHGISHRYV
SKRAAEILGKKLEELKIITCHIGNGASVAAVKYGKCVDTSMGFTPLEGL
VMGTRSGDLDPAIPFFIMEKEGISPQEMYDILNKKSGVYGLSKGFSSDM
RDNFEAALKGDEWCKLVLEIYDYRIAKYIGAYAAAMNGVDAIVFTAGVG
ENSPITREDVCKYLEFLGVKLDKQKNEETILGKEGIISTPDSRVKVLVV
PTNEELMIARDTKEIVEKIGR

In specific examples of such occurrences, the acetate kinase enzyme comprising the amino acid sequence as set forth above in SEQ ID NO: 16 may be encoded by the DNA sequence as set forth below in SEQ ID NO: 17.

(SEQ ID NO: 17)
ATGGGTAGCCATCATCATCATCATCACGGTAGCCGTGTTCTGAATATCA
ATAGCGGTAGCAGCAGCATTAAATATCAGCTGATTGAAATGGAAGGTGA
AAAAGTTCTGTGTAAAGGTATTGCAGAACGTATTGGTATTGAAGGTAGC
CGTCTGGTTCATCGTGTTGGTGATGAAAAACATGTTATTGAACGTGAAC
TGCCGGATCATGAAGAAGCACTGAAACTGATTCTGAATACCCTGGTTGA
TGAAAAACTGGGTGTTATTAAAGATCTGAAAGAAATTGACGCAGTTGGT
CATCGTGTTGTTCATGGTGGTGAACGTTTTAAAGAAAGCGTTCTGGTTG
ATGAAGAAGTTCTGAAAGCAATTGAAGAAGTTAGCCCGCTGGCACCGCT
GCATAATCCGGCAAATCTGATGGGTATTAAAGCAGCAATGAAACTGCTG
CCGGGTGTTCCGAATGTTCAAGTTTTTGATACCGCATTTCATCAGACCA
TTCCGCAGAAAGCATATCTGTATGCAATTCCGTATGAATATTACGAAAA
ATACAAAATTCGTCGCTACGGTTTTCATGGTATCAGCCATCGTTATGTT
AGCAAACGTGCAGCAGAAATTCTGGGTAAAAAACTGGAAGAACTGAAAA
TTATCACCTGTCATATTGGTAATGGTGCAAGCGTTGCAGCAGTTAAATA
TGGTAAATGTGTTGATACCAGCATGGGTTTTACCCCGCTGGAAGGTCTG
GTTATGGGTACCCGTAGCGGTGATCTGGATCCGGCAATTCCGTTTTTTA
TTATGGAAAAAGAGGGTATTAGCCCGCAGGAAATGTATGATATTCTGAA
TAAAAAAAGCGGCGTTTATGGTCTGAGCAAAGGTTTTAGCAGCGATATG
CGTGATAATTTTGAAGCAGCACTGAAAGGTGATGAATGGTGTAAACTGG
TTCTGGAAATTTATGACTACCGTATTGCAAAATACATCGGTGCATACGC
TGCAGCAATGAATGGTGTTGATGCAATTGTTTTTACCGCAGGTGTTGGT
GAAAATAGCCCGATCACCCGTGAAGATGTTTGTAAGTATCTGGAATTTC
TGGGTGTTAAACTGGATAAACAGAAAAATGAAGAGACTATTCTGGGTAA
AGAAGGTATTATTAGCACCCCGGATAGCCGTGTTAAAGTTCTGGTTGTT
CCGACCAATGAAGAACTGATGATTGCACGTGATACCAAAGAAATTGTTG
AAAAAATCGGTCGTTAA

In some occurrences of this second instance, the at least one acetate kinase enzyme is an acetate kinase enzyme based on the amino acid sequences of SEQ ID NO: 14 or 16 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 14 or 16. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences of this second instance, the at least one acetate kinase enzyme is an acetate kinase enzyme encoded by the DNA sequences of SEQ ID NO: 15 or 17 and can comprise an amino acid sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 15 or 17. These differences can be amino acid insertions, deletions, substitutions, or any combinations of such changes. In some occurrences, the sequence differences can comprise non-conservative, conservative, as well as a combination of non-conservative and conservative amino acid substitutions.

In a third instance of the second aspect, the at least one pyruvate oxidase enzyme is one or more pyruvate oxidase enzymes selected from the group consisting of wild-type pyruvate oxidase enzymes and pyruvate oxidase enzymes that are produced from the directed evolution from a wild-type pyruvate oxidase enzyme. In specific instances, the at least one pyruvate oxidase enzyme is a wild-type pyruvate oxidase enzyme having the amino acid sequence as set forth below in SEQ ID NO: 18.

(SEQ ID NO: 18)
MSDNKINIGLAVMKILESWGADTIYGIPSGTLSSLMDAMGEEENNVKFL
QVKHEEVGAMAAVMQSKFGGNLGVTVGSGGPGASHLINGLYDAAMDNIP
VVAILGSRPQRELNMDAFQELNQNPMYDHIAVYNRRVAYAEQLPKLVDE
AARMAIAKRGVAVLEVPGDFAKVEIDNDQWYSSANSLRKYEPIAPAAQD
IDAAVELLNNSKRPVIYAGIGTMGHGPAVQELARKIKAPVITTGKNFET
FEWDFEALTGSTYRVGWKPANETILEADTVLFAGSNFPFSEVEGTFRNV
DNFIQIDIDPAMLGKRHHADVAILGDAGLAIDEILNKVDAVEESAWWTA
NLKNIANWREYINMLETKEEGDLQFYQVYNAINNHADEDAIYSIDVGNS
TQTSIRHLHMTPKNMWRTSPLFATMGIAIPGGLGAKNTYPDRQVWNIIG
DGAFSMTYPDVVTNVRYNMPVINVVFSNTEYAFIKNKYEDTNKNLFGVD
FTDVDYAKIAEAQGAKGFTVSRIEDMDRVMAEAVAANKAGHTVVIDCKI
TQDRPIPVETLKLDSKLYSEDEIKAYKERYEAANLVPFREYLEAEGLES
KYIK

In specific occurrences, the wild-type pyruvate oxidase enzyme comprising the amino acid sequence as set forth above in SEQ ID NO: 18 may be encoded by the DNA sequence as set forth below in SEQ ID NO: 19.

(SEQ ID NO: 19)
ATGAGTGATAATAAAATCAACATCGGTCTGGCGGTGATGAAAATTCTGG
AAAGTTGGGGCGCAGATACCATTTATGGCATTCCGAGCGGCACCCTGAG
TAGCTTAATGGATGCCATGGGTGAAGAAGAAAACAATGTTAAGTTCCTG
CAGGTGAAGCATGAAGAAGTGGGCGCAATGGCAGCCGTTATGCAGAGTA
AATTTGGCGGTAATCTGGGTGTGACCGTTGGTAGTGGCGGTCCTGGTGC
AAGTCATCTGATTAATGGCCTGTATGATGCCGCAATGGATAATATTCCG
GTTGTGGCAATTCTGGGTAGCCGTCCTCAGCGTGAACTGAATATGGATG
CATTTCAGGAACTGAACCAGAATCCGATGTATGATCATATCGCCGTTTA
CAATCGCCGTGTGGCCTATGCAGAACAGCTGCCTAAACTGGTGGATGAA
GCCGCCCGTATGGCCATTGCTAAACGCGGTGTGGCAGTTCTGGAAGTTC
CGGGTGATTTTGCAAAAGTGGAAATTGATAACGACCAGTGGTACAGTAG
CGCAAATAGCCTGCGCAAATATGAACCGATTGCCCCGGCAGCACAGGAT
ATTGATGCAGCTGTGGAACTGCTGAATAATAGTAAACGCCCGGTGATTT
ACGCAGGTATTGGTACCATGGGCCATGGCCCTGCAGTTCAAGAACTGGC
TCGTAAAATTAAGGCACCGGTGATTACCACCGGTAAAAATTTTGAAACC
TTCGAGTGGGACTTCGAAGCCCTGACCGGTTCAACCTATCGTGTGGGTT
GGAAACCGGCAAATGAAACCATTCTGGAAGCCGATACCGTTCTGTTTGC
AGGCAGCAATTTTCCGTTTAGCGAAGTGGAAGGCACCTTTCGTAATGTT
GATAATTTCATCCAGATCGACATCGACCCGGCAATGCTGGGTAAACGCC
ATCATGCAGATGTGGCAATTCTGGGTGATGCCGGCCTGGCAATTGATGA
AATTCTGAATAAGGTGGACGCCGTGGAAGAAAGCGCCTGGTGGACCGCA
AATCTGAAAAATATTGCAAACTGGCGCGAGTATATCAACATGCTGGAAA
CCAAAGAGGAGGGTGATCTGCAGTTTTATCAGGTTTATAACGCGATCAA
CAACCACGCCGATGAAGATGCAATTTATAGCATTGACGTGGGCAATAGC
ACCCAGACCAGCATTCGTCATCTGCACATGACCCCGAAAAATATGTGGC
GCACCAGCCCGCTGTTTGCCACCATGGGTATTGCAATTCCGGGTGGCCT
GGGTGCAAAAAATACCTATCCGGATCGTCAGGTGTGGAATATTATTGGC
GATGGCGCCTTTAGTATGACCTATCCGGATGTTGTTACCAATGTTCGCT
ATAATATGCCGGTTATCAACGTGGTTTTCAGTAATACCGAGTACGCATT
TATCAAGAACAAGTACGAGGACACCAACAAAAACCTGTTTGGTGTGGAT
TTCACCGATGTGGATTATGCCAAAATCGCCGAAGCACAGGGTGCAAAAG
GCTTTACCGTGAGTCGCATTGAAGATATGGATCGTGTTATGGCCGAAGC
CGTGGCCGCAAATAAAGCCGGTCATACCGTTGTGATTGATTGTAAAATC
ACCCAGGACCGTCCGATTCCGGTTGAAACCCTGAAACTGGATAGCAAAC
TGTATAGTGAGGACGAAATCAAGGCATATAAGGAACGTTACGAGGCAGC
AAATCTGGTGCCGTTTCGTGAATATCTGGAAGCCGAAGGTCTGGAAAGC
AAATATATTAAGTAA

In specific occurrences of this third instance, the at least one pyruvate oxidase enzyme is selected from a wild-type pyruvate oxidase enzyme having the amino acid sequence as set forth below in SEQ ID NO: 20.

(SEQ ID NO: 20)
MTQGKITASAAMLNVLKTWGVDTIYGIPSGTLSSLMDALAEDKDIRFLQ
VRHEETGALAAVMQAKFGGSIGVAVGSGGPGATHLINGVYDAAMDNTPF
LAILGSRPVNELNMDAFQELNQNPMYHGIAVYNKRVAYAEQLPKVIDEA
CRAAVSKKGPAVVEIPVNFGFQEIDENSYYGSGSYERSFIAPALNEVEI
DKAVEILNKAERPVIYAGFGGVKAGEVITELSRKIKAPIITTGKNFEAF
EWNYEGLTGSAYRVGWKPANEVVFEADTVLFLGSNFPFAEVYEAFKNTE
KFIQVDIDPYKLGKRHALDASILGDAGQAAKAILDKVDAVESTPWWRAN
VKNNQNWRDYMNKLEGKTEGELQLYQVYNAINKHADQDAIYSIDVGNST
QTSTRHLHMTPKNMWRTSPLFATMGIALPGGIAAKKDNPERQVWNIMGD
GAFNMCYPDVITNVQYNLPVINVVFSNAEYAFIKNKYEDTNKHLFGVDF
TNADYAKIAEAQGAVGFTVDRIEDIDAVVAEAVKLNKEGKTVVIDARIT
QHRPLPVEVLELDPKLHSEEAIKAFKEKYEAEELVPFRLFLEEEGLQSR
AIK

In specific examples of such occurrences, the pyruvate oxidase enzyme comprising the amino acid sequence as set forth above in SEQ ID NO: 20 may be encoded by the DNA sequence as set forth below in SEQ ID NO: 21.

(SEQ ID NO: 21)
ATGACCCAGGGTAAAATTACCGCAAGTGCCGCAATGCTGAATGTGCTGA
AAACCTGGGGCGTTGATACCATTTATGGTATTCCGAGTGGTACCCTGAG
CAGCCTGATGGATGCCTTAGCAGAAGATAAAGATATCCGCTTTCTGCAG
GTGCGCCATGAAGAAACCGGCGCATTAGCCGCAGTGATGCAGGCAAAAT
TTGGTGGCAGCATTGGTGTTGCAGTGGGTAGCGGTGGTCCGGGTGCAAC
GTTTATGATGCCGCCATGGATAATACCCCGTTTCTGGCCATTCTGGGTA
GCCACCTGATCAATGGTTCGCCCTGTGAATGAACTGAATATGGATGCAT
TCCAGGAGCTGAATCAGAATCCGATGTATCATGGTATCGCCGTTTATAA
TAAGCGCGTTGCATACGCTGAACAGCTGCCGAAAGTGATTGATGAAGCA
TGCCGTGCAGCCGTGAGCAAAAAAGGCCCTGCAGTGGTTGAAATTCCGG
TTAATTTTGGCTTCCAGGAAATCGATGAGAACAGTTATTACGGCAGCGG
TAGTTATGAACGTAGCTTTATTGCCCCGGCCCTGAATGAAGTGGAAATT
GATAAAGCAGTGGAGATCCTGAACAAGGCAGAACGCCCGGTGATTTATG
CAGGCTTTGGTGGCGTGAAAGCAGGTGAAGTGATTACCGAACTGAGCCG
CAAAATTAAAGCCCCGATTATTACCACCGGCAAAAATTTTGAGGCCTTT
GAATGGAACTACGAGGGACTGACCGGCAGTGCATATCGTGTGGGTTGGA
AACCGGCAAATGAAGTGGTGTTTGAAGCCGATACCGTGCTGTTTCTGGG
TAGCAATTTTCCGTTTGCCGAAGTTTATGAGGCATTTAAAAACACCGAG
AAGTTCATCCAGGTGGATATTGATCCGTATAAGCTGGGCAAACGTCATG
CACTGGATGCAAGTATTCTGGGTGATGCAGGTCAGGCCGCAAAAGCAAT
TCTGGATAAAGTTGATGCCGTGGAAAGCACCCCGTGGTGGCGTGCAAAT
GTGAAAAATAATCAGAACTGGCGCGACTATATGAACAAACTGGAAGGCA
AAACCGAGGGTGAACTGCAGCTGTATCAGGTTTATAATGCCATTAACAA
GCACGCAGACCAGGATGCAATTTATAGTATTGACGTGGGCAACAGCACC
CAGACCAGTACACGTCATCTGCACATGACCCCGAAAAATATGTGGCGTA
CCAGCCCGCTGTTTGCAACCATGGGTATTGCCCTGCCGGGCGGTATTGC
TGCAAAAAAAGATAATCCGGAGCGTCAGGTTTGGAATATTATGGGTGAT
GGTGCCTTTAACATGTGCTATCCGGATGTGATTACCAATGTTCAGTACA
ATCTGCCGGTTATTAACGTTGTTTTCAGCAATGCCGAGTACGCATTTAT
TAAGAACAAGTACGAGGACACCAACAAGCATCTGTTTGGTGTTGATTTC
ACCAACGCCGATTATGCCAAAATTGCCGAAGCACAGGGCGCAGTTGGTT
TTACCGTGGATCGCATTGAAGATATTGACGCAGTGGTTGCCGAAGCAGT
GAAACTGAATAAAGAAGGTAAGACCGTGGTGATTGACGCCCGCATTACC
CAGCATCGCCCTTTACCTGTGGAAGTGCTGGAACTGGATCCGAAACTGC
ATAGTGAAGAAGCAATTAAGGCCTTTAAGGAGAAGTACGAAGCCGAAGA
ACTGGTGCCGTTTCGTCTGTTTCTGGAAGAAGAAGGCCTGCAGAGTCGC
GCAATTAAATAA

In specific occurrences of this third instance, the at least one pyruvate oxidase enzyme is selected from a wild-type pyruvate oxidase enzyme having the amino acid sequence as set forth below in SEQ ID NO: 22.

(SEQ ID NO: 22)
MSDKKISAGLAALKVMEGWGIDTMYGIPSGTLSGLMNAMGHPENKVKFI
QVKHEEIGAMAAVMQYKFGGNIAVTVGSGGPGATHLINGLYDAYMDHIP
VLAILGSKPVRELNMDSFQELNQNPMYDHIAVYNRRVATAEQLPHLVDD
AIRTAISKRGVAVLEVPADFGFAELDAESIYSTPLYSSGTKYKRYKPVA
PEAEEIDEAVEILKASERPVIYAGFGTQGHGDLVEELSRKIKAPVITTG
KNFDNFNWDFEALTGSAFRVGWKPANEAVLEADTVLFIGTNFPFSEVEG
TFRNVKKFIQIDANPDMLGKRHETDVAILADAGESLKALLAKVEPVADT
PWWQANIKNVQNWRDYMNKLEQKTEGPLQAYQVYNAINKLADEDAIFST
DVGDVTQLSTRHLHMNPKQMWRTSPLFATMGIALPGGIGAKNIYPDRQV
WNIIGDGAFSMTYPDVVTSVRYDMPMINVIFTNTEYGFIKNKYEDTNEY
NFGVDFTDVDYAKVGEAQGAIGLTVNRIEDIDRVMQEAVDYYKQGRVVV
VDAKITKDRPIPVETLKLDTNLYSEDVVKAYKEKYEAEALVPFREFLEG
EGLKSIYIKEDNDNKFSF

In specific examples of such occurrences, the pyruvate oxidase enzyme comprising the amino acid sequence as set forth above in SEQ ID NO: 22 may be encoded by the DNA sequence as set forth below in SEQ ID NO: 23.

(SEQ ID NO: 23)
ATGAGTGATAAAAAAATCAGCGCGGGCCTGGCAGCACTGAAAGTTATGG
AAGGTTGGGGTATTGATACCATGTATGGTATTCCGAGCGGTACCCTGAG
CGGTTTAATGAATGCAATGGGCCATCCGGAAAATAAAGTTAAATTCATC
CAGGTGAAGCACGAGGAAATTGGCGCAATGGCAGCCGTTATGCAGTATA
AATTTGGTGGCAATATCGCGGTTACCGTTGGTAGCGGTGGCCCTGGTGC
AACCCACCTGATCAATGGTCTGTATGATGCCTATATGGACCATATTCCG
GTTCTGGCAATTCTGGGTAGTAAACCGGTTCGCGAACTGAATATGGATA
GTTTTCAGGAACTGAACCAGAACCCGATGTATGATCATATTGCCGTTTA
CAACCGCCGCGTTGCAACCGCAGAACAGTTACCTCATCTGGTGGATGAT
GCAATTCGCACCGCCATTAGTAAACGCGGTGTGGCTGTTCTGGAAGTTC
CGGCAGATTTTGGTTTTGCCGAACTGGATGCCGAAAGCATTTATAGCAC
CCCGCTGTATAGTAGTGGCACCAAATATAAACGCTACAAGCCGGTTGCA
CCGGAAGCCGAGGAAATTGATGAAGCCGTGGAAATTCTGAAGGCAAGCG
AACGTCCGGTTATTTATGCCGGTTTTGGTACCCAGGGCCATGGTGATCT
GGTGGAAGAACTGAGTCGCAAAATTAAAGCACCGGTGATTACCACCGGT
AAAAATTTTGATAACTTCAACTGGGACTTCGAGGCACTGACCGGCTCAG
CATTTCGCGTTGGTTGGAAACCGGCCAATGAAGCCGTGCTGGAAGCCGA
CACAGTTCTGTTTATTGGCACCAATTTCCCGTTTAGTGAGGTGGAAGGC
ACCTTTCGCAATGTTAAAAAATTCATCCAGATCGACGCAAACCCGGATA
TGCTGGGTAAACGTCATGAAACCGATGTTGCCATTCTGGCAGATGCCGG
TGAAAGCCTGAAAGCCCTGCTGGCAAAAGTTGAACCGGTGGCAGATACC
CCGTGGTGGCAGGCAAATATTAAAAATGTGCAGAACTGGCGCGACTATA
TGAATAAACTGGAACAGAAGACCGAGGGCCCGCTGCAGGCATACCAGGT
GTACAATGCAATTAATAAGCTGGCCGATGAGGATGCCATTTTTAGTACC
GATGTGGGTGATGTTACCCAGCTGAGCACCCGTCATCTGCACATGAATC
CGAAACAGATGTGGCGTACCAGTCCGCTGTTTGCAACCATGGGCATTGC
CCTGCCGGGTGGTATTGGAGCAAAAAATATTTACCCGGACCGTCAGGTT
TGGAATATTATTGGCGATGGCGCATTTAGCATGACCTATCCGGATGTTG
TTACCAGTGTTCGTTATGATATGCCGATGATTAACGTGATCTTCACCAA
TACCGAGTACGGTTTTATCAAGAACAAGTACGAGGACACCAACGAATAT
AACTTCGGCGTGGATTTTACCGATGTGGATTATGCCAAAGTGGGCGAAG
CCCAGGGCGCAATCGGTCTGACCGTGAACAGAATTGAAGATATTGACCG
CGTGATGCAGGAAGCCGTGGATTATTATAAACAGGGTCGCGTTGTTGTG
GTTGATGCAAAAATTACCAAGGACCGCCCGATTCCGGTTGAAACCCTGA
AACTGGATACCAATCTGTATAGTGAGGACGTTGTTAAGGCCTATAAGGA
AAAATACGAGGCAGAAGCACTGGTTCCGTTTCGCGAATTTCTGGAAGGT
GAAGGTCTGAAAAGTATCTATATCAAGGAGGACAACGACAACAAGTTCA
GTTTTTAA

In specific occurrences of this third instance, the at least one pyruvate oxidase enzyme is the wild-type pyruvate oxidase enzyme that comprises the amino acid sequence as set forth below in SEQ ID NO: 24.

(SEQ ID NO: 24)
MTVGKTKVSTASLKVLAGWGIDTIYGIPSGTLAPLMEALGEQEETDIKF
LQVKHEEVGAMAAVMQWKFGGKLGVCVGSGGPGASHLINGLYDAAMDNT
PVLAILGSPPQRELNMDAFQELNQNPMYDHIAVYNRRVAYAEQLPKLID
DAIRTAISKRGVAVLEVPGDFGYKEIANDAFYSSGHSYRDYVSSAINEV
DIDAAVEVLNKSKRAVIYAGIGTMGHGPAVQELSRKIKAPIITTAKNFE
TFDYDFEGLTGSTYRVGWKPANEAVKEADTVLFVGSNFPFAEVEGTFSN
VENFIQIDNNPTMLGKRHNADVAILGDAGEAVQMLLEKVAPVEESAWWN
ANLKNIQNWRDYMTKLETKENGPLQLYQVYNAINKYADEDAIYSIDVGN
TTQTSIRHLHMTPKNMWRTSPLFASMGIALPGGIGAKNVYPERQVFNLM
GDGAFSMNYQDIVTNVRYNMPVINVVFTNTEYGFIKNKYEDTNTNTFGT
EFTDVDYAMIGEAQGAVGFTVSRIEDMDQVMAAAVKANKEGKTVVIDAK
ITKDRPIPVETLKLDPALYSEEEIKAYKERYEAEELVPFSEFLKAEGLE
SKVAK

In some occurrences, the at least one pyruvate oxidase enzyme is a pyruvate oxidase enzyme based on the amino acid sequences of SEQ ID NO: 18, 20, 22, or 24 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 18, 20, 22, or 24. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one pyruvate oxidase enzyme is a pyruvate oxidase enzyme encoded by the DNA sequences of SEQ ID NO: 19, 21, or 23 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 19, 21, or 23. These differences can be amino acid insertions, deletions, substitutions, or any combinations of such changes. In some occurrences, the sequence differences can comprise non-conservative, conservative, as well as a combination of non-conservative and conservative amino acid substitutions.

In a fourth instance of the second aspect, the at least one catalase enzyme is selected from the group consisting of wild-type catalase enzymes and catalase enzymes that are produced from the directed evolution from a commercially available, wild-type catalase enzyme, including the catalase enzyme commercially available as product number 11650645103 from Roche Diagnostics International Ltd. In specific occurrences of this fourth instance, the at least one catalase enzyme is the wild-type catalase enzyme that comprises the amino acid sequence as set forth below in SEQ ID NO: 52.

(SEQ ID NO: 52)
MSEKSAADQIVDRGMRPKLSGNTTRHNGAPVPSENISATAGPQGPNVLN
DIHLIEKLAHFNRENVPERIPHAKGHGAFGELHITEDVSEYTKADLFQP
GKVTPLAVRFSTVAGEQGSPDTWRDVHGFALRFYTEEGNYDIVGNNTPT
FFLRDGMKFPDFIHSQKRLNKNGLRDADMQWDFWTRAPESAHQVTYLMG
DRGTPKTSRHQDGFGSHTFQWINAEGKPVWVKYHFKTRQGWDCFTDAEA
AKVAGENADYQREDLYNAIENGDFPIWDVKVQIMPFEDAENYRWNPFDL
TKTWSQKDYPLIPVGYFILNRNPRNFFAQIEQIALDPGNIVPGVGLSPD
RMLQARIFAYADQQRYRIGANYRDLPVNRPINEVNTYSREGSMQYIFDA
EGEPSYSPNRYDKGAGYLDNGTDSSSNHTSYGQADDIYVNPDPHGTDLV
RAAYVKHQDDDDFIQPGILYREVLDEGEKERLADNISNAMQGISEATEP
RVYDYWNNVDENLGARVKELYLQKKA

In specific examples of such occurrences, the catalase enzyme comprising the amino acid sequence as set forth above in SEQ ID NO: 52 may be encoded by the DNA sequence as set forth below in SEQ ID NO: 53.

(SEQ ID NO: 53)
ATGAGCGAAAAGAGCGCGGCGGACCAAATTGTTGATCGTGGTATGCGTC
CGAAACTGAGCGGCAACACCACCCGTCACAACGGTGCGCCGGTTCCGAG
CGAAAACATCAGCGCGACCGCGGGTCCGCAGGGTCCGAACGTGCTGAAC
GACATCCACCTGATTGAGAAGCTGGCGCACTTCAACCGTGAAAACGTTC
CGGAGCGTATTCCGCACGCGAAAGGTCACGGCGCGTTTGGTGAACTGCA
CATCACCGAAGACGTGAGCGAGTACACCAAGGCGGACCTGTTCCAACCG
GGTAAAGTGACCCCGCTGGCGGTTCGTTTTAGCACCGTTGCGGGCGAGC
AAGGTAGCCCGGACACCTGGCGTGATGTTCACGGTTTCGCGCTGCGTTT
TTACACCGAGGAAGGTAACTACGATATTGTGGGCAACAACACCCCGACC
TTCTTTCTGCGTGACGGTATGAAGTTCCCGGATTTTATCCACAGCCAGA
AGCGTCTGAACAAAAACGGCCTGCGTGACGCGGATATGCAGTGGGACTT
TTGGACCCGTGCGCCGGAAAGCGCGCACCAAGTTACCTATCTGATGGGT
GACCGTGGTACCCCGAAGACCAGCCGTCACCAGGATGGTTTCGGCAGCC
ACACCTTTCAATGGATCAACGCGGAGGGCAAACCGGTGTGGGTTAAGTA
CCACTTCAAAACCCGTCAGGGTTGGGACTGCTTTACCGATGCGGAAGCG
GCGAAGGTGGCGGGCGAGAACGCGGACTACCAACGTGAAGATCTGTATA
ACGCGATCGAGAACGGTGACTTCCCGATTTGGGATGTGAAAGTTCAGAT
CATGCCGTTCGAAGATGCGGAGAACTACCGTTGGAACCCGTTTGACCTG
ACCAAGACCTGGAGCCAAAAAGATTATCCGCTGATCCCGGTTGGTTACT
TTATTCTGAACCGTAACCCGCGTAACTTCTTTGCGCAGATCGAACAAAT
TGCGCTGGACCCGGGCAACATTGTGCCGGGTGTTGGTCTGAGCCCGGAC
CGTATGCTGCAGGCGCGTATTTTCGCGTACGCGGATCAGCAACGTTATC
GTATCGGTGCGAACTACCGTGACCTGCCGGTTAACCGTCCGATTAACGA
AGTGAACACCTATAGCCGTGAGGGCAGCATGCAATACATCTTTGATGCG
GAGGGTGAACCGAGCTACAGCCCGAACCGTTATGACAAGGGTGCGGGCT
ATCTGGACAACGGCACCGACAGCAGCAGCAACCACACCAGCTATGGTCA
GGCGGACGATATCTACGTTAACCCGGACCCGCACGGTACCGATCTGGTT
CGTGCGGCGTATGTGAAGCACCAGGACGATGACGATTTCATCCAACCGG
GCATTCTGTACCGTGAGGTGCTGGACGAGGGTGAAAAAGAGCGTCTGGC
GGATAACATTAGCAACGCGATGCAAGGTATCAGCGAAGCGACCGAGCCG
CGTGTTTACGACTATTGGAACAACGTGGATGAAAACCTGGGCGCGCGTG
TGAAAGAGCTGTACCTGCAGAAGAAAGCGTAA

In specific examples of such occurrences, the catalase enzyme comprising the amino acid sequence as set forth above in SEQ ID NO: 52 may be encoded by the DNA sequence as set forth below in SEQ ID NO: 54.

(SEQ ID NO: 54)
ATGTCCGAAAAATCAGCGGCGGATCAGATTGTAGACCGCGGAATGCGTC
CGAAACTGTCCGGAAACACCACCCGCCACAACGGAGCGCCGGTGCCGTC
CGAGAACATTAGCGCGACCGCGGGCCCGCAGGGCCCGAACGTGCTCAAT
GATATCCATCTCATCGAAAAACTCGCGCACTTTAACCGCGAAAACGTGC
CGGAGCGTATTCCTCATGCGAAAGGCCACGGCGCATTCGGTGAACTGCA
TATTACCGAGGATGTAAGCGAATACACCAAAGCGGATCTGTTCCAGCCT
GGTAAAGTTACCCCCCTGGCAGTGCGCTTCAGCACAGTGGCAGGTGAAC
AGGGCAGCCCAGACACCTGGCGCGATGTTCACGGCTTCGCACTGCGCTT
CTACACCGAAGAGGGCAACTACGATATTGTAGGTAACAACACCCCGACC
TTCTTCCTGCGTGATGGCATGAAATTCCCCGATTTCATTCATTCACAGA
AACGTCTCAACAAAAACGGTCTGCGCGACGCGGATATGCAGTGGGACTT
CTGGACCCGCGCGCCTGAATCCGCGCACCAGGTAACCTACCTGATGGGT
GATCGCGGTACCCCTAAAACCAGCCGCCATCAGGATGGCTTCGGCAGCC
ACACCTTCCAGTGGATTAACGCAGAAGGTAAACCGGTGTGGGTGAAATA
CCATTTCAAAACCCGCCAGGGCTGGGACTGCTTCACCGACGCGGAAGCG
GCGAAAGTGGCGGGCGAAAACGCAGATTACCAGCGCGAAGATCTCTACA
ACGCAATCGAAAACGGCGATTTCCCGATTTGGGATGTTAAAGTGCAGAT
TATGCCTTTCGAGGACGCGGAAAACTACCGCTGGAACCCGTTCGACCTG
ACCAAAACCTGGTCCCAGAAAGACTACCCGCTGATTCCGGTTGGTTACT
TCATTCTGAACCGCAACCCGCGCAACTTCTTCGCACAGATTGAGCAGAT
CGCGCTGGACCCGGGCAACATTGTGCCTGGCGTTGGCCTGAGCCCGGAT
CGCATGCTCCAGGCGCGTATTTTCGCGTACGCGGATCAGCAGCGTTACC
GCATTGGCGCAAACTACCGCGATCTGCCGGTAAACCGTCCGATTAACGA
AGTTAACACCTACTCACGCGAAGGTAGCATGCAGTACATTTTCGATGCA
GAGGGCGAACCTAGCTACTCACCTAACCGCTACGATAAAGGCGCGGGCT
ACCTGGATAACGGTACTGACAGCAGCAGCAACCACACCAGCTACGGCCA
GGCAGACGATATTTACGTTAACCCGGATCCGCACGGCACCGATCTGGTG
CGTGCTGCATACGTTAAACACCAGGACGACGATGATTTCATTCAGCCGG
GCATTCTATACCGCGAGGTTCTGGACGAAGGCGAGAAAGAACGATTGGC
GGATAACATTAGCAACGCTATGCAGGGCATTTCCGAGGCAACCGAACCG
CGCGTTTACGATTACTGGAACAACGTGGACGAGAACCTCGGCGCACGCG
TTAAAGAACTGTACCTCCAGAAAAAAGCTTAA

In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is at least about 85%, 86%, 87%, 88%, 89%, 90%, 91%, 92%, 93%, 94%, 95%, 96%, 97%, 98%, or 99% identical to the reference sequence SEQ ID NO: 52. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to the reference sequence of the catalase enzyme commercially available as product number 11650645103 from Roche Diagnostics International Ltd. In some occurrences, the at least one catalase enzyme is encoded by DNA sequences that are at least about 85%, 86%, 87%, 88%, 89%, 90%, 91%, 92%, 93%, 94%, 95%, 96%, 97%, 98%, or 99% identical to SEQ ID NO: 53 or 54. These differences can be amino acid insertions, deletions, substitutions, or any combinations of such changes. In some occurrences, the sequence differences can comprise non-conservative, conservative, as well as a combination of non-conservative and conservative amino acid substitutions.

In a fifth instance of the second aspect, the at least one uridine phosphorylase enzyme is one or more uridine phosphorylase enzymes selected from the group consisting of wild-type uridine phosphorylase enzymes and uridine phosphorylase enzymes that are produced from the directed evolution from a commercially available, wild-type uridine phosphorylase enzyme. In specific instances, the at least one uridine phosphorylase enzyme is selected from a wild-type uridine phosphorylase enzyme having the amino acid sequence as set forth below in SEQ ID NO: 25.

(SEQ ID NO: 25)
MSKSDVFHLGLTKNDLQGATLAIVPGDPDRVEKIAALMDKPVKLASHRE
FTTWRAELDGKPVIVCSTGIGGPSTSIAVEELAQLGIRTFLRIGTTGAI
QPHINVGDVLVTTASVRLDGASLHFAPLEFPAVADFECTTALVEAAKSI
GATTHVGVTASSDTFYPGQERYDTYSGRVVRHFKGSMEEWQAMGVMNYE
MESATLLTMCASQGLRAGMVAGVIVNRTQQEIPNAETMKQTESHAVKIV
VEAARRLL

In specific occurrences, the wild-type uridine phosphorylase enzyme comprising the amino acid sequence as set forth above in SEQ ID NO: 25 may be encoded by the DNA sequence as set forth below in SEQ ID NO: 26.

(SEQ ID NO: 26)
ATGTCCAAGTCTGATGTTTTTCATCTCGGCCTCACTAAAAACGATTTAC
AAGGGGCTACGCTTGCCATCGTCCCTGGCGACCCGGATCGTGTGGAAAA
GATCGCCGCGCTGATGGATAAGCCGGTTAAGCTGGCATCTCACCGCGAA
TTCACTACCTGGCGTGCAGAGCTGGATGGTAAACCTGTTATCGTCTGCT
CTACCGGTATCGGCGGCCCGTCTACCTCTATTGCTGTTGAAGAGCTGGC
ACAGCTGGGCATTCGCACCTTCCTGCGTATCGGTACAACGGGCGCTATT
CAGCCGCATATTAATGTGGGTGATGTCCTGGTTACCACGGCGTCTGTCC
GTCTGGATGGCGCGAGCCTGCACTTCGCACCGCTGGAATTCCCGGCTGT
CGCTGATTTCGAATGTACGACTGCGCTGGTTGAAGCTGCGAAATCCATT
GGCGCGACAACTCACGTTGGCGTGACAGCTTCTTCTGATACCTTCTACC
CAGGTCAGGAACGTTACGATACTTACTCTGGTCGCGTAGTTCGTCACTT
TAAAGGTTCTATGGAAGAGTGGCAGGCGATGGGCGTAATGAACTATGAA
ATGGAATCTGCAACCCTGCTGACCATGTGCGCAAGTCAGGGCCTGCGTG
CCGGTATGGTAGCGGGTGTTATCGTTAACCGCACCCAGCAAGAGATCCC
GAATGCTGAGACGATGAAACAAACCGAAAGCCATGCGGTGAAAATCGTG
GTGGAAGCGGCGCGTCGTCTGCTGTAA

In specific occurrences of this fifth instance, the at least one uridine phosphorylase enzyme is a wild-type uridine phosphorylase enzyme that comprises the amino acid sequence as set forth below in SEQ ID NO: 27.

(SEQ ID NO: 27)
MHHHHHHGGSGMSKSDVFHLGLTKNDLQGATLAIVPGDPDRVEKIAALM
DKPVKLASHREFTTWRAELDGKPVIVCSTGIGGPSTSIAVEELAQLGIR
TFLRIGTTGAIQPHINVGDVLVTTASVRLDGASLHFAPLEFPAVADFEC
TTALVEAAKSIGATTHVGVTASSDTFYPGQERYDTYSGRVVRHFKGSME
EWQAMGVMNYEMESATLLTMCASQGLRAGMVAGVIVNRTQQEIPNAETM
KQTESHAVKIVVEAARRLL

In specific occurrences of this fifth instance, the at least one uridine phosphorylase enzyme is a uridine phosphorylase enzyme having the amino acid sequence as set forth below in SEQ ID NO: 28.

(SEQ ID NO: 28)
MHHHHHHGGSGMSKSDVFHLGLTKNDLQGATLAIVPGDPDRVEKIAALM
DKPVKLASHREFTTWRAELDGKPVIVCSTGIGGPSTSIAVEILAQLGIR
TFLRIGTTGAIQPHINVGDVLVTTASVRLDGASLHFAPLEFPAVADFEC
TTALVEAAKSIGATTHVGVTASSDTFYPGQERYDTYSGRVVRHFKGSME
EWQAMGVMNYEMESATLLTMCASQGLRAGMVAGVIVNRTQQEIPNAETM
KQTESHAVKIVVEAARRLL

In specific examples of such occurrences, the uridine phosphorylase enzyme comprising the amino acid sequence as set forth above in SEQ ID NO: 28 may be encoded by the DNA sequence as set forth below in SEQ ID NO: 29.

(SEQ ID NO: 29)
ATGCACCATCATCATCATCATGGCGGTAGCGGCATGTCCAAGTCTGATG
TTTTTCATCTCGGCCTCACTAAAAACGATTTACAAGGGGCTACGCTTGC
CATCGTCCCTGGCGACCCGGATCGTGTGGAAAAGATCGCCGCGCTGATG
GATAAGCCGGTTAAGCTGGCATCTCACCGCGAATTCACTACCTGGCGTG
CAGAGCTGGATGGTAAACCTGTTATCGTCTGCTCTACCGGTATCGGCGG
CCCGTCTACCTCTATTGCTGTTGAAATTCTGGCACAGCTGGGCATTCGC
ACCTTCCTGCGTATCGGTACAACGGGCGCTATTCAGCCGCATATTAATG
TGGGTGATGTCCTGGTTACCACGGCGTCTGTCCGTCTGGATGGCGCGAG
CCTGCACTTCGCACCGCTGGAATTCCCGGCTGTCGCTGATTTCGAATGT
ACGACTGCGCTGGTTGAAGCTGCGAAATCCATTGGCGCGACAACTCACG
TTGGCGTGACAGCTTCTTCTGATACCTTCTACCCAGGTCAGGAACGTTA
CGATACTTACTCTGGTCGCGTAGTTCGTCACTTTAAAGGTTCTATGGAA
GAGTGGCAGGCGATGGGCGTAATGAACTATGAAATGGAATCTGCAACCC
TGCTGACCATGTGCGCAAGTCAGGGCCTGCGTGCCGGTATGGTAGCGGG
TGTTATCGTTAACCGCACCCAGCAAGAGATCCCGAATGCTGAGACGATG
AAACAAACCGAAAGCCATGCGGTGAAAATCGTGGTGGAAGCGGCGCGTC
GTCTGCTGTAA

In specific occurrences of this fifth instance, the at least one uridine phosphorylase enzyme is a uridine phosphorylase enzyme having the amino acid sequence as set forth below in SEQ ID NO: 30.

(SEQ ID NO: 30)
MHHHHHHGGSGMSESDSFHLGLTKNDLQGATLAILPGDPDRVEKIAALM
DKPVKLASWREFTTWRAELDGKPVIVCSTGIGGPSTSIAVEILAQLGIR
TFLRIGTTGAIQPHINVGDVLVTTASVRLDGASLHFAPLEFPAVADFEC
TTALVEAAKSIGATTHVGVTASSDTFYPGQERYDTYSGRVVRHFKGSME
EWQRMGVMNYEMESATLLTMCASQGLRAGMVAGVIVNRTQQELPNAESM
KQTESHMVKIVVEAARRLL

In specific occurrences of this fifth instance, the at least one uridine phosphorylase enzyme is a uridine phosphorylase enzyme having the amino acid sequence as set forth below in SEQ ID NO: 31.

(SEQ ID NO: 31)
MHHHHHHGGSGMSESDSFHMGLTKNDLQGATLAILPGDPARVEKIAALM
DNPVKLASWREFTTWRAELDGKPVIVCSTGIGGPSTSIAVEILAQLGIR
TFLRIGTTGAIQGHINVGDVLVTTASVRLDGASGHFAPMEFPAVADFEC
TTALVEAAKSIGATTHVGVTASSDTFYPGQERYDTYSGRVVRRFQGSME
EWQEMGVMNYEMESATLLTMCASQGLRAGMVAGVIVNRTQQELPNAESM
KQVESHMVKIVVEAARRLL

In specific occurrences of this fifth instance, the at least one uridine phosphorylase enzyme is a uridine phosphorylase enzyme having the amino acid sequence as set forth below in SEQ ID NO: 45

(SEQ ID NO: 45)
MHHHHHHGGSGMSESDTFHMGLTKNDLQGATLAIVPGDPARVEKIAALM
DNPVKLGSWREFITWRAELDGKPVIVCSTGIGGPSTSIAVEILAQLGIR
TFLRIGTTGAIQGHINVGDVLVTTASVRLDGASGHFAPYEFPAVADFEC
TTALVEAAKSIGATTHVGVTASSDTFYPGQERYDTYSGRVVRRFQGSME
EWREMGVMNYEMESATLLTMCASQGLRAGMVAGVIVNRTQQELPNAESM
KQVESHMVKIVVEAARRLL

In specific occurrences of this fifth instance, the at least one uridine phosphorylase enzyme is a uridine phosphorylase enzyme having the amino acid sequence as set forth below in SEQ ID NO: 46

(SEQ ID NO: 46)
MHHHHHHGGSGMSESDTFHMGLTKNDLQGATLAILPGDPARVEKIAALM
DNPVKLGSWREFITWRAELDGKPVIVCSTGIGGPSTSIAVEILAQLGIR
TFLRIGTIGAIQGHINVGDVLVTTASVRLDGASGHFAPYEFPAVADFEC
TTALVEAAKSIGATTHVGVTASSDTFYPGQERYDTYSGRVVRRFQGSME
EWRVMGVMNYEMESATLLTMCASQGLRAGMVAGVIVNRTQQELPNAEDM
PQVESHMVKIVVEAARRLL

In specific occurrences of this fifth instance, the at least one uridine phosphorylase enzyme is a uridine phosphorylase enzyme having the amino acid sequence as set forth below in SEQ ID NO: 47

(SEQ ID NO: 47)
MHHHHHHGGSGMSESDTFHMGLTKNDLQGATLAILPGDPARVEKIAALM
DNPVKLGQWREFITWRAELDGKPVIVCSTGIGGPSTSIAVEILAQLGIR
TFLRIGTTGAIQGHINVGDVLVTTASVRLDGASGHFAPYEFPAVADFEC
TTALVEAAKSCGATTHVGVTASSDTFYPGQERYDTYSGRVVRRFRGSME
EWRVMGVMNYEMESATLLTMCASQGLRAGMVAGVIVNRTQQELPNAESM
PQVESFMVKIVVEAARRLL

In specific occurrences of this fifth instance, the at least one uridine phosphorylase enzyme is a uridine phosphorylase enzyme having the amino acid sequence as set forth below in SEQ ID NO: 48

(SEQ ID NO: 48)
MHHHHHHGGSGMSESDSFHMGLTKNDLQGATLAIVPGDPARVEKIAALM
DNPVKLGQWREFITWRAELDGKPVIVCSTGIGGPSTSIAVEILAQLGIR
TFLRIGTTGAIQGHINVGDVLVTTASVRLDGASGHFAPMEFPAVADFEC
TTALVEAAKSCGATTHVGVTASSDTFYPGQERYDTYSGRVVRRFQGSME
EWREMGVMNYEMESATLLTMCASQGLRAGMVAGVIVNRTQQELPNAESM
KQVESHMVKIVVEAARRLL

In specific occurrences of this fifth instance, the at least one uridine phosphorylase enzyme is a uridine phosphorylase enzyme having the amino acid sequence as set forth below in SEQ ID NO: 49

(SEQ ID NO: 49)
MHHHHHHGGSGMSESDSFHMGLTKNDLQGATLAIVPGDPARVEKIAALM
DNPVKLGSWREFITWRAELDGKPVIVCSTGIGGPSTSIAVEILAQLGIR
TFLRIGTTGAIQGHIGVGDVLVTTASVRLDGASGHFAPYEFPAVADFEC
TTALVEAAKSCGATTHVGVTASSDTFYPGQERYDTYSGRVVRRFRGSME
EWREMGVMNYEMESATLLTMCAVQGLRAGMVAGVIVNRTQQELPNAEDM
KQVESHMVKIVVEAARRLL

In specific occurrences of this fifth instance, the at least one uridine phosphorylase enzyme is a uridine phosphorylase enzyme having the amino acid sequence as set forth below in SEQ ID NO: 50

(SEQ ID NO: 50)
MHHHHHHGGSGMSESDSFHMGLTKNDLQGATLAILPGDPARVEKIAALM
DNPVKLGSWREFITWRAELDGKPVIVCSTGIGGPSTSIAVEILAQLGIR
TFLRIGTTGAIQGHIGVGDVLVTTASVRLDGASGHFAPYEFPAVADFEC
TTALVEAAKSIGATTHVGVTASSDTFYPGQERYDTYSGRVVRRFQGSME
EWREMGVMNYEMESATLLTMCASQGLRAGMVAGVIVNRTQQELPNAEDM
PQVESHMVKIVVEAARRLL

In specific occurrences of this fifth instance, the at least one uridine phosphorylase enzyme is a uridine phosphorylase enzyme having the amino acid sequence as set forth below in SEQ ID NO: 51

(SEQ ID NO: 51)
MHHHHHHGGSGMSESDSFHMGLTKNDLQGATLAIVPGDPARVEKIAALM
DNPVKLGSWREFITWRAELDGKPVIVCSTGIGGPSTSIAVEILAQLGIR
TFLRIGTIGAIQGHINVGDVLVTTASVRLDGASGHFAPMEFPAVADFEC
TTALVEAAKSIGATTHVGVTASSDTFYPGQERYDTYSGRVVRRFRGSME
EWREMGVMNYEMESATLLTMCASQGLRAGMVAGVIVNRTQQELPEAEDM
PQVESHMVKIVVEAARRLL

In some occurrences, the at least one uridine phosphorylase enzyme is a uridine phosphorylase enzyme based on the amino acid sequences of SEQ ID NO: 25, 27, 28, 30, 31, 45, 46, 47, 48, 49, 50, or 51 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 25, 27, 28, 30, 31, 45, 46, 47, 48, 49, 50, or 51. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one uridine phosphorylase enzyme is a uridine phosphorylase enzyme encoded by the DNA sequences of SEQ ID NO: 26 or 29 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 26 or 29. These differences can be amino acid insertions, deletions, substitutions, or any combinations of such changes. In some occurrences, the sequence differences can comprise non-conservative, conservative, as well as a combination of non-conservative and conservative amino acid substitutions.

In a sixth instance of the second aspect, the at least one ribokinase enzyme is one or more ribokinase enzymes selected from the group consisting of wild-type ribokinase enzymes and ribokinase enzymes that are produced from the directed evolution from a commercially available, wild-type ribokinase enzyme. In specific occurrences, the at least one ribokinase enzyme is a wild-type ribokinase enzyme that comprises the amino acid sequence as set forth below in SEQ ID NO: 32.

(SEQ ID NO: 32)
MQNAGSLVVLGSINADHILNLQSFPTPGETVTGNHYQVAFGGKGANQAV
AAGRSGANIAFIACTGDDSIGESVRQQLATDNIDITPVSVIKGESTGVA
LIFVNGEGENVIGIHAGANAALSPALVEAQRERIANASALLMQLESPLE
SVMAAAKIAHQNKTIVALNPAPARELPDELLALVDIITPNETEAEKLTG
IRVENDEDAAKAAQVLHEKGIRTVLITLGSRGVWASVNGEGQRVPGFRV
QAVDTIAAGDTFNGALITALLEEKPLPEAIRFAHAAAAIAVTRKGAQPS
VPWREEIDAFLDRQR

In specific occurrences, the wild-type ribokinase enzyme comprising the amino acid sequence set forth above as SEQ ID NO: 32 may be encoded by the DNA sequence as set forth below in SEQ ID NO: 33.

(SEQ ID NO: 33)
ATGCAAAACGCAGGCAGCCTCGTTGTTCTTGGCAGCATTAATGCTGACC
ACATTCTTAATCTTCAATCTTTTCCTACTCCAGGCGAAACCGTAACCGG
TAACCACTATCAGGTTGCATTTGGCGGCAAAGGCGCGAATCAGGCTGTG
GCTGCTGGGCGTAGCGGTGCGAATATCGCGTTTATTGCCTGTACGGGTG
ATGACAGCATTGGTGAGAGCGTTCGCCAGCAGCTCGCCACTGATAACAT
TGATATTACTCCGGTCAGCGTGATCAAAGGCGAATCAACAGGTGTGGCG
CTGATTTTTGTTAATGGCGAAGGTGAGAATGTCATCGGTATTCATGCCG
GCGCTAATGCTGCCCTTTCCCCGGCGCTGGTGGAAGCGCAACGTGAGCG
TATTGCCAACGCGTCAGCATTATTAATGCAGCTGGAATCACCACTCGAA
AGTGTGATGGCAGCGGCGAAAATCGCCCATCAAAATAAGACTATCGTTG
CGCTTAACCCGGCTCCGGCTCGCGAACTTCCTGACGAACTGCTGGCGCT
GGTGGACATTATTACGCCAAACGAAACGGAAGCAGAAAAGCTCACCGGT
ATTCGTGTTGAAAATGATGAAGATGCAGCGAAGGCGGCGCAGGTACTGC
ATGAAAAAGGTATCCGTACTGTACTGATTACTTTAGGAAGTCGTGGTGT
ATGGGCTAGCGTGAATGGTGAAGGTCAGCGCGTTCCTGGATTCCGGGTG
CAGGCTGTCGATACCATTGCTGCCGGAGATACCTTTAACGGTGCGTTAA
TCACGGCATTGCTGGAAGAAAAACCATTGCCAGAGGCGATTCGTTTTGC
CCATGCTGCCGCTGCGATTGCCGTAACACGTAAAGGCGCACAACCTTCC
GTACCGTGGCGTGAAGAGATCGACGCATTTTTAGACAGGCAGAGGTAA

In some occurrences, the at least one ribokinase enzyme is a ribokinase enzyme based on the amino acid sequences of SEQ ID NO: 32 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 32. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one ribokinase enzyme is a ribokinase enzyme encoded by the DNA sequences of SEQ ID NO: 33 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 33. These differences can be amino acid insertions, deletions, substitutions, or any combinations of such changes. In some occurrences, the sequence differences can comprise non-conservative, conservative, as well as a combination of non-conservative and conservative amino acid substitutions.

In a seventh instance of the second aspect, the at least one phosphopentomutase enzyme is one or more phosphopentomutase enzymes selected from the group consisting of wild-type phosphopentomutase enzymes and phosphopentomutase enzymes that are produced from the directed evolution from a commercially available, wild-type phosphopentomutase enzyme. In specific occurrences, the at least one phosphopentomutase enzyme is a wild-type phosphopentomutase enzyme that comprises the amino acid sequence as set forth below in SEQ ID NO: 34.

(SEQ ID NO: 34)
MKRAFIMVLDSFGIGATEDAERFGDVGADTLGHIAEACAKGEADNGRKG
PLNLPNLTRLGLAKAHEGSTGFIPAGMDGNAEVIGAYAWAHEMSSGKDT
PSGHWEIAGVPVLFEWGYFSDHENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRHDLAVEPPAPTVLQK
LVDEKHGQVVSVGKIADIYANCGITKKVKATGLDALFDATIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMF

In specific occurrences, the wild-type phosphopentomutase enzyme comprising the amino acid sequence as set forth above in SEQ ID NO: 34 may be encoded by the DNA sequence as set forth below in SEQ ID NO: 35.

(SEQ ID NO: 35)
ATGAAACGTGCATTTATTATGGTGCTGGACTCATTCGGCATCGGCGCTA
CAGAAGATGCAGAACGCTTTGGTGACGTCGGGGCTGACACCCTGGGTCA
TATCGCAGAAGCTTGTGCCAAAGGCGAAGCTGATAACGGTCGTAAAGGC
CCGCTCAATCTGCCAAATCTGACCCGTCTGGGGCTGGCGAAAGCACACG
AAGGTTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTTGAGTGGG
GATATTTCTCCGATCACGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCACGACCTGGCTGTTGAGCCGCCAGCACCGACCGTGCTGCAGAAA
CTGGTTGATGAAAAACACGGCCAGGTGGTTTCTGTCGGTAAAATTGCGG
ACATCTACGCCAACTGCGGTATCACCAAAAAAGTGAAAGCGACTGGCCT
GGACGCGCTGTTTGACGCCACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCTAA

In specific occurrences of this seventh instance, the at least one phosphopentomutase enzyme is a phosphopentomutase enzyme comprising the amino acid sequence as set forth below in SEQ ID NO: 36.

(SEQ ID NO: 36)
MKRAFIMVLDSFGIGATEDAERFGDVGADTLGHIAEACAKGEADNGRKG
PLNLPNLTRLGLAKAHEGSTGFIPAGMDGNAEVIGAYAWAHEMSSGKDT
PSGHWEIAGVPVLFEWGYFSDHENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRRDLAVEPPAPTVLQK
LVDEKHGQVVSVGKIADIYANCGITKKVKATGLDALFDATIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMFHHHHHH

In specific examples of such occurrences, the phosphopentomutase enzyme comprising the amino acid sequence as set forth above in SEQ ID NO: 36 may be encoded by the DNA sequence as set forth below in SEQ ID NO: 37.

(SEQ ID NO: 37)
ATGAAACGTGCATTTATTATGGTGCTGGACTCATTCGGCATCGGCGCTA
CAGAAGATGCAGAACGCTTTGGTGACGTCGGGGCTGACACCCTGGGTCA
TATCGCAGAAGCTTGTGCCAAAGGCGAAGCTGATAACGGTCGTAAAGGC
CCGCTCAATCTGCCAAATCTGACCCGTCTGGGGCTGGCGAAAGCACACG
AAGGTTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTTGAGTGGG
GATATTTCTCCGATCACGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCGTGACCTGGCTGTTGAGCCGCCAGCACCGACCGTGCTGCAGAAA
CTGGTTGATGAAAAACACGGCCAGGTGGTTTCTGTCGGTAAAATTGCGG
ACATCTACGCCAACTGCGGTATCACCAAAAAAGTGAAAGCGACTGGCCT
GGACGCGCTGTTTGACGCCACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCCATC
ATCATCACCATCATTAA

In specific occurrences of this seventh instance, the at least one phosphopentomutase enzyme is a phosphopentomutase enzyme comprising the amino acid sequence as set forth below in SEQ ID NO: 38.

(SEQ ID NO: 38)
MKRAFIMVLDSFGIGATEDAEKFGDVGADTLGHIAEACAKGEADNGRKG
PLNVPNLTRLGLAKAHEGSTGFIPAGMDGNAEVIGAYAWAHEMSSGKDT
PSGHWEIAGVPVLFEWGYFSDLENSFPQELLDKLVERANLPGYLGNCHS
SGTVILDQLGEEHMKTGKPIFYTSADSVFQIACHEETFGLDKLYELCEI
AREELTNGGYNIGRVIARPFIGDKAGNFQRTGNRRDLAVEPPAPTVLQK
LVDEKHGQVVGVGKIADIYANCGITKKVKATGLDALFDTTIKEMKEAGD
NTIVFTNFVDFDSSWGHRRDVAGYAAGLELFDRRLPELMSLLRDDDILI
LTADHGCDPTWTGTDHTREHIPVLVYGPKVKPGSLGHRETFADIGQTLA
KYFGTSDMEYGKAMFHHHHHH

In specific examples of such occurrences, the phosphopentomutase enzyme comprising the amino acid sequence as set forth above in SEQ ID NO: 38 may be encoded by the DNA sequence as set forth below in SEQ ID NO: 39.

(SEQ ID NO: 39)
ATGAAACGTGCATTTATTATGGTGCTGGACTCATTCGGCATCGGCGCTA
CAGAAGATGCAGAAAAGTTTGGTGACGTCGGGGCTGACACCCTGGGTCA
TATCGCAGAAGCTTGTGCCAAAGGCGAAGCTGATAACGGTCGTAAAGGC
CCGCTCAATGTGCCAAATCTGACCCGTCTGGGGCTGGCGAAAGCGCACG
AAGGCTCTACCGGTTTCATTCCGGCGGGAATGGACGGCAACGCTGAAGT
TATCGGCGCGTACGCATGGGCGCACGAAATGTCATCCGGTAAAGATACC
CCGTCTGGTCACTGGGAAATTGCCGGTGTCCCGGTTCTGTTCGAGTGGG
GATATTTCTCCGATCTGGAAAACAGCTTCCCGCAAGAGCTGCTGGATAA
ACTGGTCGAACGCGCTAATCTGCCGGGTTACCTCGGTAACTGCCACTCT
TCCGGTACGGTCATTCTGGATCAACTGGGCGAAGAGCACATGAAAACCG
GCAAGCCGATTTTCTATACCTCCGCTGACTCCGTGTTCCAGATTGCCTG
CCATGAAGAAACTTTCGGTCTGGATAAACTCTACGAACTGTGCGAAATC
GCCCGTGAAGAGCTGACCAACGGCGGCTACAATATCGGTCGTGTTATCG
CTCGTCCGTTTATCGGCGACAAAGCCGGTAACTTCCAGCGTACCGGTAA
CCGTCGTGACCTGGCTGTTGAGCCGCCAGCACCGACCGTGCTGCAGAAA
CTGGTTGATGAAAAACACGGCCAGGTGGTAGGCGTCGGTAAAATTGCGG
ACATCTACGCCAACTGCGGTATCACCAAAAAAGTGAAAGCGACTGGCCT
GGACGCGCTGTTTGACACTACCATCAAAGAGATGAAAGAAGCGGGTGAT
AACACCATCGTCTTCACCAACTTCGTTGACTTCGACTCTTCCTGGGGCC
ACCGTCGCGACGTCGCCGGTTATGCCGCGGGTCTGGAACTGTTCGACCG
CCGTCTGCCGGAGCTGATGTCTCTGCTGCGCGATGACGACATCCTGATC
CTCACCGCTGACCACGGTTGCGATCCGACCTGGACCGGTACTGACCACA
CGCGTGAACACATTCCGGTACTGGTATATGGCCCGAAAGTAAAACCGGG
CTCACTGGGTCATCGTGAAACCTTCGCGGATATCGGCCAGACTCTGGCA
AAATATTTTGGTACTTCTGATATGGAATATGGCAAAGCCATGTTCCATC
ATCATCACCATCATTAA

In some occurrences, the at least one phosphopentomutase enzyme is a phosphopentomutase enzyme based on the amino acid sequences of SEQ ID NO: 34, 36, or 38 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 34, 36, or 38. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one phosphopentomutase enzyme is a phosphopentomutase enzyme encoded by the DNA sequences of SEQ ID NO: 35, 37, or 39 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 35, 37, or 39. These differences can be amino acid insertions, deletions, substitutions, or any combinations of such changes. In some occurrences, the sequence differences can comprise non-conservative, conservative, as well as a combination of non-conservative and conservative amino acid substitutions.

In an eighth instance of the second aspect, the at least one sucrose phosphorylase enzyme is one or more sucrose phosphorylase enzymes selected from the group consisting of wild-type sucrose phosphorylase enzymes and sucrose phosphorylase enzymes that are produced from the directed evolution from a commercially available, wild-type sucrose phosphorylase enzyme. In specific occurrences, the at least one sucrose phosphorylase enzyme is a wild-type sucrose phosphorylase enzyme that comprises the amino acid sequence as set forth below in SEQ ID NO: 40.

(SEQ ID NO: 40)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTPQQVDIDTDSEMGWNYLLSILDQLSQSHVSQIRLDAVGY
GAKEKNSSCFMTPKTFKLIERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNVGRDINRHYYSAAEVASEVERPVVQALNALGRFRNTLSAFDGEFS
YSNADGVLTMTWADDATRATLTFAPKANSNGASVARLEWTDAAGEHATD
DLIANPPVVA

In specific occurrences of this eighth instance, the at least one sucrose phosphorylase enzyme is a sucrose phosphorylase enzyme comprising the amino acid sequence as set forth below in SEQ ID NO: 41.

(SEQ ID NO: 41)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTRQQVDIDTDSEMGWNYLLSILDQLSQSHVSQIRLDAVGY
GAKEKNSSCFLTPKTFKLVERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNSGRGINRHYYSAAEVASEVERPVVQALNALGRFRNTLSAFDGEFS
YSNADGVLTMTWADDATRATLTFAPKANSNGASVARLEWTDAAGEHATD
DLIANPPVVA

In specific occurrences, the sucrose phosphorylase enzyme comprising the amino acid sequence as set forth above in SEQ ID NO: 41 may be encoded by the DNA sequence as set forth below in SEQ ID NO: 42.

(SEQ ID NO: 42)
ATGAAGAACAAAGTTCAACTGATTACCTATGCGGATCGTCTGGGTGACG
GCACCCTGAAGAGCATGACCGAGACCCTGCGTAAACACTTCGAGGGTGT
TTATGAAGGCGTGCACATCCTGCCGTTCTTTACCCCGTTCGATGGTGCG
GACGCGGGCTTTGATCCGGTTGACCACACCAAAGTGGACCCGCGTCTGG
GTAGCTGGGACGATGTGGCGGAACTGAGCACCACCCACGATATCATGGT
TGACACCATTGTGAACCACATGAGCTGGGAGAGCGAACAGTTCCAAGAT
GTTATGGCGAAGGGCGAGGACAGCGAATACTATCCGATGTTCCTGACCA
TGAGCAGCATTTTTCCGGATGGTGTGACCGAGGAAGACCTGACCGCGAT
CTATCGTCCGCGTCCGGGTCTGCCGTTCACCCACTATAACTGGGGTGGC
AAAACCCGTCTGGTTTGGACCACCTTTACCCGTCAGCAAGTGGACATTG
ATACCGACAGCGAGATGGGTTGGAACTACCTGCTGAGCATCCTGGATCA
GCTGAGCCAAAGCCACGTTAGCCAAATTCGTCTGGACGCGGTGGGTTAT
GGCGCGAAGGAGAAAAACAGCAGCTGCTTCCTGACCCCGAAGACCTTTA
AACTGGTCGAACGTATTAAGGCGGAGGGTGAAAAACGTGGCCTGGAGAC
CCTGATCGAAGTTCACAGCTACTATAAGAAACAGGTGGAGATTGCGAGC
AAGGTGGATCGTGTTTACGACTTTGCGATCCCGGGTCTGCTGCTGCATG
CGCTGGAATTTGGCAAAACCGATGCGCTGGCGCAATGGATTGACGTTCG
TCCGAACAACGCGGTGAACGTTCTGGATACCCACGACGGTATCGGCGTT
ATCGATATTGGTAGCGATCAGATGGACCGTAGCCTGGCGGGTCTGGTGC
CGGATGAGGAAGTTGACGCGCTGGTTGAGAGCATCCACCGTAACAGCAA
GGGTGAAAGCCAGGAAGCGACCGGCGCGGCGGCGAGCAACCTGGACCTG
TACCAAGTTAACTGCACCTACTATGCGGCGCTGGGTAGCGACGATCAGA
AATATATTGCGGCGCGTGCGGTGCAGTTCTTTATGCCGGGCGTGCCGCA
AGTTTACTATGTGGGTGCGCTGGCGGGCAGCAACGATATGGACCTGCTG
AAGCGTACCAACAGTGGTCGTGGCATCAACCGTCACTACTATAGCGCGG
CGGAAGTGGCGAGCGAGGTGGAACGTCCGGTGGTTCAGGCGCTGAACGC
GCTGGGCCGTTTCCGTAACACCCTGAGCGCGTTCGATGGTGAATTTAGC
TACAGCAACGCGGACGGCGTTCTGACCATGACCTGGGCGGATGATGCGA
CCCGTGCGACCCTGACCTTTGCGCCGAAGGCGAACAGCAACGGTGCGAG
CGTGGCGCGTCTGGAGTGGACCGATGCGGCGGGTGAACATGCGACCGAC
GATCTGATCGCGAACCCGCCGGTGGTTGCGTAA

In specific occurrences of this eighth instance, the at least one sucrose phosphorylase enzyme is a sucrose phosphorylase enzyme comprising the amino acid sequence as set forth below in SEQ ID NO: 43.

(SEQ ID NO: 43)
MKNKVQLITYADRLGDGTLKSMTETLRKHFEGVYEGVHILPFFTPFDGA
DAGFDPVDHTKVDPRLGSWDDVAELSTTHDIMVDTIVNHMSWESEQFQD
VMAKGEDSEYYPMFLTMSSIFPDGVTEEDLTAIYRPRPGLPFTHYNWGG
KTRLVWTTFTRQQVDIDTDSEMGWNYLLSILDQLSQSHVSQIRLDAVGY
GAKEKNSSCFLTPKTFKLVERIKAEGEKRGLETLIEVHSYYKKQVEIAS
KVDRVYDFAIPGLLLHALEFGKTDALAQWIDVRPNNAVNVLDTHDGIGV
IDIGSDQMDRSLAGLVPDEEVDALVESIHRNSKGESQEATGAAASNLDL
YQVNCTYYAALGSDDQKYIAARAVQFFMPGVPQVYYVGALAGSNDMDLL
KRTNSGRGINRHYYSAAEVASEVERPVVQALNALGRFRNTLSAFDGEFS
YSNADGVLTMTWADDATRATLTFAPKANSNGASVARLEWTDAAGEHATD
DLIANPPVVAGQTGHHHHHH

In specific examples of such occurrences, the sucrose phosphorylase enzyme comprising the amino acid sequence as set forth above in SEQ ID NO: 43 may be encoded by the DNA sequence as set forth below in SEQ ID NO: 44.

(SEQ ID NO: 44)
ATGAAGAACAAAGTTCAACTGATTACCTATGCGGATCGTCTGGGTGACG
GCACCCTGAAGAGCATGACCGAGACCCTGCGTAAACACTTCGAGGGTGT
TTATGAAGGCGTGCACATCCTGCCGTTCTTTACCCCGTTCGATGGTGCG
GACGCGGGCTTTGATCCGGTTGACCACACCAAAGTGGACCCGCGTCTGG
GTAGCTGGGACGATGTGGCGGAACTGAGCACCACCCACGATATCATGGT
TGACACCATTGTGAACCACATGAGCTGGGAGAGCGAACAGTTCCAAGAT
GTTATGGCGAAGGGCGAGGACAGCGAATACTATCCGATGTTCCTGACCA
TGAGCAGCATTTTTCCGGATGGTGTGACCGAGGAAGACCTGACCGCGAT
CTATCGTCCGCGTCCGGGTCTGCCGTTCACCCACTATAACTGGGGTGGC
AAAACCCGTCTGGTTTGGACCACCTTTACCCGTCAGCAAGTGGACATTG
ATACCGACAGCGAGATGGGTTGGAACTACCTGCTGAGCATCCTGGATCA
GCTGAGCCAAAGCCACGTTAGCCAAATTCGTCTGGACGCGGTGGGTTAT
GGCGCGAAGGAGAAAAACAGCAGCTGCTTCCTGACCCCGAAGACCTTTA
AACTGGTCGAACGTATTAAGGCGGAGGGTGAAAAACGTGGCCTGGAGAC
CCTGATCGAAGTTCACAGCTACTATAAGAAACAGGTGGAGATTGCGAGC
AAGGTGGATCGTGTTTACGACTTTGCGATCCCGGGTCTGCTGCTGCATG
CGCTGGAATTTGGCAAAACCGATGCGCTGGCGCAATGGATTGACGTTCG
TCCGAACAACGCGGTGAACGTTCTGGATACCCACGACGGTATCGGCGTT
ATCGATATTGGTAGCGATCAGATGGACCGTAGCCTGGCGGGTCTGGTGC
CGGATGAGGAAGTTGACGCGCTGGTTGAGAGCATCCACCGTAACAGCAA
GGGTGAAAGCCAGGAAGCGACCGGCGCGGCGGCGAGCAACCTGGACCTG
TACCAAGTTAACTGCACCTACTATGCGGCGCTGGGTAGCGACGATCAGA
AATATATTGCGGCGCGTGCGGTGCAGTTCTTTATGCCGGGCGTGCCGCA
AGTTTACTATGTGGGTGCGCTGGCGGGCAGCAACGATATGGACCTGCTG
AAGCGTACCAACAGTGGTCGTGGCATCAACCGTCACTACTATAGCGCGG
CGGAAGTGGCGAGCGAGGTGGAACGTCCGGTGGTTCAGGCGCTGAACGC
GCTGGGCCGTTTCCGTAACACCCTGAGCGCGTTCGATGGTGAATTTAGC
TACAGCAACGCGGACGGCGTTCTGACCATGACCTGGGCGGATGATGCGA
CCCGTGCGACCCTGACCTTTGCGCCGAAGGCGAACAGCAACGGTGCGAG
CGTGGCGCGTCTGGAGTGGACCGATGCGGCGGGTGAACATGCGACCGAC
GATCTGATCGCGAACCCGCCGGTGGTTGCGGGCCAAACTGGCCACCATC
ACCATCACCATTAGTAA

In some occurrences, the at least one sucrose phosphorylase enzyme is a sucrose phosphorylase enzyme based on the amino acid sequences of SEQ ID NO: 40, 41, or 43 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 40, 41, or 43. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some occurrences, the at least one sucrose phosphorylase enzyme is a sucrose phosphorylase enzyme encoded by the DNA sequences of SEQ ID NO: 42 or 44 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 42 or 44. These differences can be amino acid insertions, deletions, substitutions, or any combinations of such changes. In some occurrences, the sequence differences can comprise non-conservative, conservative, as well as a combination of non-conservative and conservative amino acid substitutions.

In a third aspect of this first embodiment, the reacting is conducted in the presence of at least one solvent. In instances of this aspect, the at least one solvent is selected from aqueous solvents, organic solvents and mixtures thereof. In particular instances of this aspect, the at least one solvent is water. In other particular instances of this aspect, the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof. In still further instances of this aspect, the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

In a fourth aspect of this first embodiment, the process further comprises isolating uridine.

Aspects of this embodiment disclose a process for preparing uridine:

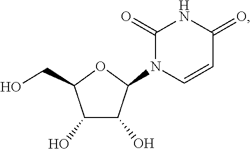 [SMILES: O=c1ccn([C@@H]2O[C@H](CO)[C@@H](O)[C@H]2O)c(=O)[nH]1]

the process comprising reacting ribose with uracil in the presence of an enzyme, which is selected from the group consisting of S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes, and mixtures thereof, to form uridine. In such aspects, the enzyme, S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes are as described above.

### EXAMPLES

### Example 1: Biocatalytic Synthesis of Uridine

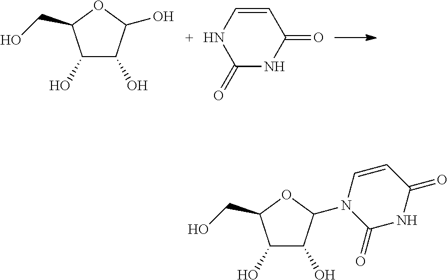 [SMILES: OC[C@H]1OC(O)[C@H](O)[C@@H]1O ; O=c1cc[nH]c(=O)[nH]1 ; O=c1ccn(C2O[C@H](CO)[C@@H](O)[C@H]2O)c(=O)[nH]1]

In a 100 mL reactor, water (25 mL) was charged. Pyruvic acid (2.75 g, 31.2 mmol), K₂HPO₄ (0.784 g, 4.5 mmol), and aqueous 1M MgCl₂ solution (0.5 mL, 0.5 mmol) were charged. The pH was determined to be 1.6, and the pH was raised with 8N KOH (approximately 3.6 mL) to 7.1. D-ribose (3.75 g, 25.0 mmol), thiamine pyrophosphate (“TPP”, 28.8 mg, 0.125 mmol), ATP (75.5 mg, 0.25 mmol) were added to the reactor. The pH was adjusted with 8N KOH to 7.0. A polymeric antifoam agent (to prevent formation of excessive foam; commercially available as GLANAPON 2000 KONZ from Bussetti, 20 ul) was added. Catalase enzyme (commercially available as product number 11650645103 from Roche Diagnostics International Ltd., 28 mg), pyruvate oxidase enzyme (SEQ ID NO: 20, 28 mg), acetate kinase enzyme (SEQ ID NO: 14, 113 mg), uridine phosphorylase enzyme (SEQ ID NO: 27, 113 mg), and MTR-kinase enzyme (SEQ ID NO: 55, 113 mg) were added to the reactor. Then, uracil (1.80 g, 16.1 mmol) was added to the reactor. The mixture was agitated with supplying air at 25° C. After 41 hours, the mixture was assayed by HPLC to find that uridine (3.57 g, 14.6 mmol) was formed, which is approximately 71.4 g/L concentration of uridine.

### Example 2: Biocatalytic Synthesis of Uridine

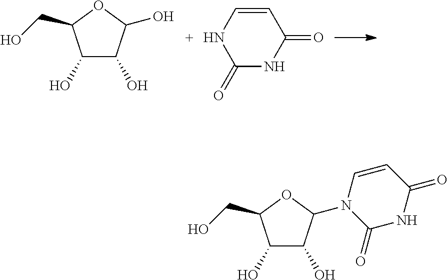 [SMILES: OC[C@H]1OC(O)[C@H](O)[C@@H]1O ; O=c1cc[nH]c(=O)[nH]1 ; O=c1ccn(C2O[C@H](CO)[C@@H](O)[C@H]2O)c(=O)[nH]1]

In a well of 24-well plate (approximately 10 mL size), a mixture of D-ribose (214 mg, 1.43 mmol) , uracil (80 mg, 0.713 mmol), pyruvic acid (157 mg, 1.78 mmol), K₂HPO₄ (43.5 mg, 0.25 mmol), MgCl₂ hexahydrate (4.1 mg, 0.02 mmol), TPP (3.3 mg), ATP (4.3 mg), flavin adenine dinucleotide (“FAD”, 0.3 mg) in water (total 2 mL volume) was added. The pH was adjusted with aqueous KOH to 7.0). Catalase enzyme (commercially available as product number 11650645103 from Roche Diagnostics International Ltd., 0.8 mg), pyruvate oxidase enzyme (SEQ ID NO: 18, 0.8 mg), acetate kinase enzyme (SEQ ID NO: 16, 0.8 mg), uridine phosphorylase enzyme (SEQ ID NO: 27, 0.8 mg), and MTR-kinase enzyme (SEQ ID NO: 1, 16 mg) were added to the reactor. The mixture was shaken at 25° C. After 116 hours, the conversion of uracil to uridine was observed (162 mg of uridine).

### Example 3: Biocatalytic Synthesis of Uridine

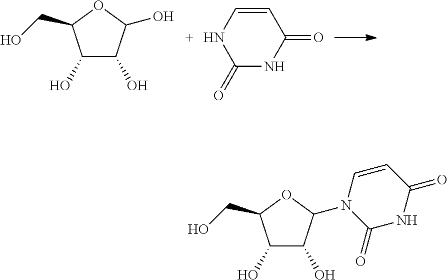 [SMILES: OC[C@H]1OC(O)[C@H](O)[C@@H]1O ; O=c1cc[nH]c(=O)[nH]1 ; O=c1ccn(C2O[C@H](CO)[C@@H](O)[C@H]2O)c(=O)[nH]1]

In a 50 mL reactor, 568 mg triethanolamine was dissolved in 36 mL water for 100 mM. MgCl₂ hexahydrate (38 mg, to 5 mM) was added. Uracil (266 mg, 2.373 mmol), sucrose (3249 mg, 9.49 mmol), and D-ribose (713 mg, 4.75 mmol) were added. Propionyl phosphate monoammonium salt (974 mg, 5.70 mmol) was added to the suspension, giving a pH of 5.3. The pH was raised to 7.42 with 5N KOH. ATP (13.1 mg, 0.024 mmol) was added, followed by acetate kinase enzyme (SEQ ID NO: 16, 2.7 mg), uridine phosphorylase enzyme (SEQ ID NO: 27, 8.0 mg), and sucrose phosphorylase enzyme (SEQ ID NO: 43, 0.8 mg). The pH was observed to be 7.35. MTR kinase enzyme (SEQ ID NO: 13, 80 mg) was dissolved in 3 mL of 100 mM triethanolamine/HCl buffer (pH 7.5) and added to the stirring mixture. The mixture was stirred at 25° C., while maintaining a pH of 7.35 to 7.45 using 4N KOH. After 22 hours, conversion of uracil to uridine was observed (524 mg of uridine).

### Example 4: Biocatalytic Synthesis of Uridine

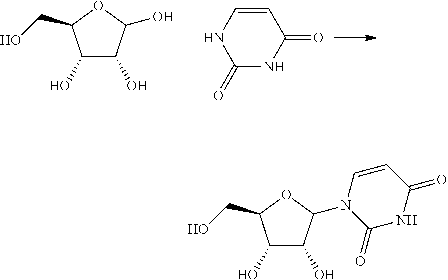 [SMILES: OC[C@H]1OC(O)[C@H](O)[C@@H]1O ; O=c1cc[nH]c(=O)[nH]1 ; O=c1ccn(C2O[C@H](CO)[C@@H](O)[C@H]2O)c(=O)[nH]1]

Triethanolamine (TEoA) (59.6 mg) was dissolved in 4 mL water. Propionyl phosphate monoammonium salt (231 mg, 1.349 mmol) was added. The pH was adjusted from 4.5 to 7.5 with 5N KOH. Manganese chloride tetrahydrate (4.0 mg) and MgCl₂ (1.9 mg) were added. Uracil (56 mg, 0.500 mmol), sucrose (1026 mg, 3.00 mmol), D-ribose (169 mg, 1.124 mmol), and adenosine 5′-triphosphate disodium salt (5.51 mg, 9.99 μmol) were added. The pH was adjusted from 7.33 to 7.58 before addition of enzymes. Phosphopentomutase enzyme (SEQ ID NO: 34, 2.2 mg), uridine phosphorylase enzyme (SEQ ID NO: 27, 1.1 mg) and sucrose phosphorylase enzyme (SEQ ID NO: 43, 1.1 mg) were added. A solution of 2.8 mg/mL each of acetate kinase enzyme (SEQ ID NO: 16) and ribokinase enzyme (SEQ ID NO: 32) was prepared in a 100 mM, pH 7.5 TEoA/acetic acid buffer with 5 mM MnCl₂. 100 μL of this solution was added, and the suspension was stirred at 24° C. The pH was maintained between 6.9 and 7.6 with periodic addition of 5N KOH. After 23.5 hours, HPLC assay showed 82% conversion of uracil to uridine (100 mg, 0.410 mmol).

It will be appreciated that various of the above-discussed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

1. A process for preparing uridine: 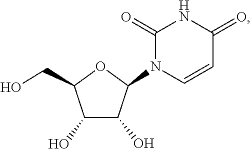 [SMILES: O=c1ccn([C@@H]2O[C@H](CO)[C@@H](O)[C@H]2O)c(=O)[nH]1]
the process comprising reacting ribose with uracil in the presence of at least one enzyme to form uridine.

2. The process according to claim 1, wherein uracil is provided in an amount in a range of from about 0.4 to about 1.2 equivalents with respect to the amount of ribose.

3. The process according to claim 1, wherein the at least one enzyme is selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme, and mixtures thereof.

4. The process according to claim 1, wherein the at least one enzyme is a mixture of at least two enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme.

5. The process according to claim 4, wherein the at least one enzyme is a mixture of at least three enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme.

6. The process according to claim 4, wherein the at least one enzyme is a mixture of at least four enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme.

7. The process according to claim 4, wherein the at least one enzyme is a mixture of at least five enzymes selected from the group consisting of at least one S-methyl-5-thioribose kinase enzyme, at least one acetate kinase enzyme, at least one pyruvate oxidase enzyme, at least one catalase enzyme, at least one uridine phosphorylase enzyme, at least one ribokinase enzyme, at least one phosphopentomutase enzyme, and at least one sucrose phosphorylase enzyme.

8. The process according to claim 4, wherein the at least one enzyme is a mixture of at least one S-methyl-5-thioribose kinase enzyme, at least one acetate kinase enzyme, at least one pyruvate oxidase enzyme, at least one catalase enzyme, and at least one uridine phosphorylase enzyme.

9. The process according to claim 4, wherein the at least one enzyme is a mixture of at least one S-methyl-5-thioribose kinase enzyme, at least one acetate kinase enzyme, at least one uridine phosphorylase enzyme, and at least one sucrose phosphorylase enzyme.

10. The process according to claim 4, wherein the at least one enzyme is a mixture of at least one acetate kinase enzyme, at least one pyruvate oxidase enzyme, at least one catalase enzyme, at least one uridine phosphorylase enzyme, at least one ribokinase enzyme, and at least one phosphopentomutase enzyme.

11. The process according to claim 4, wherein the at least one enzyme is a mixture of at least one S-methyl-5-thioribose kinase enzyme, at least one acetate kinase enzyme, at least one uridine phosphorylase enzyme, at least one ribokinase enzyme, at least one phosphopentomutase enzyme, and at least one sucrose phosphorylase enzyme.

12. The process according to claim 4, wherein the at least one enzyme is a mixture of at least one acetate kinase enzyme, at least one uridine phosphorylase enzyme, and at least two enzymes selected from the group consisting of at least one S-methyl-5-thioribose kinase enzyme, at least one pyruvate oxidase enzyme, at least one catalase enzyme, at least one ribokinase enzyme, at least one phosphopentomutase enzyme, and at least one sucrose phosphorylase enzyme.

13. A process for preparing uridine: 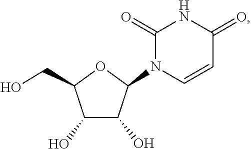 [SMILES: O=c1ccn([C@@H]2O[C@H](CO)[C@@H](O)[C@H]2O)c(=O)[nH]1]
the process comprising reacting ribose with uracil in the presence of an enzyme, which is selected from the group consisting of S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes, and mixtures thereof, to form uridine.

14. The process according to claim 13, wherein the enzyme is a mixture of at least two enzymes selected from the group consisting of S-methyl-5-thioribose kinase enzymes, acetate kinase enzymes, pyruvate oxidase enzymes, catalase enzymes, uridine phosphorylase enzymes, ribokinase enzymes, phosphopentomutase enzymes, and sucrose phosphorylase enzymes, and mixtures thereof.

15. The process according to claim 1, wherein the reacting is conducted in the presence of at least one solvent selected from aqueous solvents, organic solvents and mixtures thereof.

16. The process according to claim 15, wherein the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

17. The process according to claim 15, wherein the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

18. The process according to claim 13, wherein the reacting is conducted in the presence of at least one solvent selected from aqueous solvents, organic solvents and mixtures thereof.

19. The process according to claim 18, wherein the at least one solvent is selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.

20. The process according to claim 18, wherein the at least one solvent is water and at least one organic solvent selected from the group consisting of DME, anisole, tert-butanol, tert-amyl alcohol, acetone, 1,2-propylene carbonate, 1,3-dioxolane, acetonitrile, ethyl acetate, methyl tert-butyl ether, cyclopentyl methyl ether, chlorobenzene, isopropanol, isopropyl acetate, 2-butanone, and mixtures thereof.
